SEQUENCE LISTING (1 sequence)

SEQ ID NO: 1 - protein, 9 aa, Artificial Sequence
  AH1 peptide antigen
SPSYVYHGF

(19) United States
(12) Patent Application Publication
(10) Pub. No.: US 2023/0091468 A1
(43) Pub. Date: Mar. 23, 2023

(54) SUSTAINED IMMUNOTHERAPY

(71) Applicant: Fusion Pharmaceuticals Inc., Hamilton (CA)

(72) Inventors: Eric Steven Burak, Cambridge (CA); julie Metcalf, Toronto (CA); Natalie Grinshtein, Oakville (CA); Meiduo Hu, Maple (CA); John Fitznaurice valliant, Ancaster (CA); Sonal Patel, Hamilton (CA)

(21) Appl. No.: 17/791,824

(22) PCT Filed: Jan. 8, 2021
(86) PCT No.: PCT/US2021/012656

§ 371 Date: Jul. 8, 2022

Related U.S. Application Data
(60) Provisional application No. 63/037,520, filed on Jun. 10, 2020.
(60) Provisional application No. 62/959,879, filed on Jan. 10, 2020.

(51) Int. Cl. A61K 51/10; A61K 45/06; A61P 35/04

(52) U.S. Cl. CPC A61K 51/1096; A61K 51/1027; A61K 45/06; A61P 35/04

(57) ABSTRACT

Methods of inducing CD8+ T cell infiltration into a tumor in a patient in need thereof comprising administering a radioimmunoconjugate that is capable of binding a target expressed by at least some cells in a tumor.

### RELATED APPLICATIONS

The present application claims priority to U.S. Provisional Patent Application No. 62/959,879, filed Jan. 10, 2020, and U.S. Provisional Patent Application No. 63/037,520, filed Jun. 10, 2020, the entire contents of each of which are hereby incorporated by reference for all purposes.

### SEQUENCE LISTING

The present application contains a Sequence Listing which has been submitted electronically in ASCII format and is hereby incorporated by reference in its entirety. Said ASCII copy, created on Jan. 4, 2021, is named FPI_009_Sequence_Listing_ST25.txt and is 480 bytes in size.

### BACKGROUND

Numerous therapeutic agents have been evaluated for treatment of cancer. However, many therapeutic agents demonstrate limited treatment efficacy when used as a monotherapy or display a maximum tolerated dose not suitable for treatment. Existing treatment failures include cancerous cells that persist even after treatment due to an inability of cytotoxic therapeutics to kill all viable tumor cells or, in the case of passive or targeted immunotherapeutics, a failure to elicit and to recruit sufficient numbers of cytotoxic T cells. Additionally, cancer cells can metastasize to form secondary tumors and/or undergo genetic rearrangement that allows them to resist the therapeutic effects of anti-cancer agent treatments that previously resulted in improvements in patient tumor volumes and even apparent complete tumor regression.

Accordingly, there is a need for therapeutic agents and methods of treatment that provide a sustained form of anti-cancer therapy that can be used alone or in combination with other anti-cancer therapeutic agents to achieve a complete and/or persistent anti-cancer therapeutic effect. There is also an extreme need for treatments effective with so-called cold tumors, which are resistant to current immunotherapeutic modalities.

### SUMMARY

Presently disclosed methods can be used to induce infiltration of a CD8⁺ T cell population into the core of a tumor, even in tumors that are typically not highly responsive to immunotherapies (such as cold tumors). In accordance with presently disclosed methods, a patient in need thereof is administered a radioimmunoconjugate that is capable of binding a target expressed by at least some cells in a tumor. In some embodiments, the CD8+ T cell population that infiltrates into the tumor persists in the patient and may therefore act to prevent metastases from forming and/or reduce the likelihood of recurrences.

In one aspect, provided are methods of inducing CD8+ T cell infiltration into a tumor in a subject in need thereof, wherein the method comprises a step of administering to the subject a radioimmunoconjugate or a pharmaceutical composition thereof, wherein the radioimmunoconjugate comprises the following structure:

A-L-B Formula I-a

wherein
A is a metal complex of a chelating moiety, wherein the metal complex comprises Actinium-225 (²²⁵Ac) or a progeny thereof,
L is a linker, and
B is a targeting moiety capable of binding a first tumor-associated antigen expressed by at least some cells in the tumor;

with the proviso that if A-L- is a metal complex of Compound 1 as shown below, then B is not AVE1642

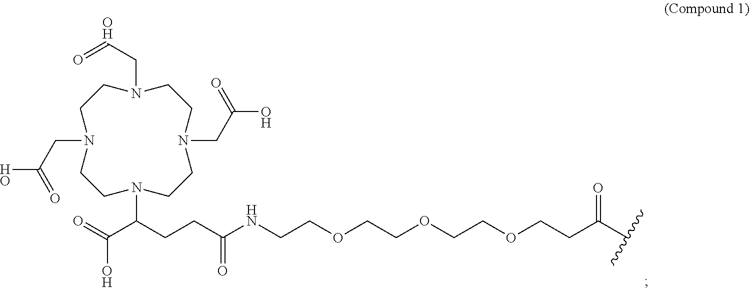

wherein said administering of said radioimmunoconjugate results in infiltration of a CD8⁺ T cell population into the core of the tumor; wherein said CD8⁺ T cell population comprises CD8+ T cells expressing a T-cell receptor (TCR) specific for a second tumor-associated antigen expressed by at least some cells in the tumor; and wherein the CD8+ T cell is capable of preferentially killing a cell expressing the second tumor-associated antigen.

In some embodiments, the CD8+ T cell population is detectable in the core of the tumor at a level greater than a reference level, e.g., at least two-fold, at least three-fold, at least four-fold, or at least five-fold greater than the reference level.

In some embodiments, the CD8+ T cell population represents at least 5%, at least 7.5%, at least 10%, at least 12.5%, or at least 15% of cells (e.g., of viable cells) in the core of the tumor.

In some embodiments, the CD8+ T cells represent at least 15%, at least 20%, at least 25%, at least 30%, at least 45%, at least 50%, at least 55%, at least 60%, at least 65%, or at least 70% of said CD8+ T cell population.

In some embodiments, the CD8+ T cells are detectable in the subject at least 10 days, at least 15 days, at least 20 days, at least 25 days, at least 30 days, at least 35 days, or at least 40 days after the step of administering.

In some embodiments, the first tumor-associated antigen is different than the second tumor-associated antigen. In some embodiments, the second tumor-associated antigen is a neoantigen.

In some embodiments, the tumor is a primary tumor. In some embodiments, the tumor is a secondary tumor.

In some embodiments, the tumor is not highly immunogenic. For example, the tumor may be moderately immunogenic or immunologically cold.

In some embodiments, the tumor is at least 100 mm³, at least 150 mm³, or at least or about 175 mm³ in volume at the time of administering.

In some embodiments, the tumor is a solid tumor.

For example, the solid tumor may be a sarcoma, e.g., a sarcoma selected from the group consisting of angiosarcoma or hemangioendothelioma, astrocytoma, chondrosarcoma, Ewing's sarcoma, fibrosarcoma, glioma, leiomyosarcoma, liposarcoma, malignant fibrous histiocytoma (MFH), mesenchymous or mixed mesodermal tumor, mesothelial sarcoma or mesothelioma, myxosarcoma, osteosarcoma, rhabdomyosarcoma, and synovial sarcoma. In some embodiments, the sarcoma is osteosarcoma.

For example, the solid tumor may be a carcinoma, e.g., a carcinoma selected from the group consisting of adenoid cystic carcinoma, adrenocortical carcinoma, bladder cancer, breast cancer, cervical cancer, colorectal cancer, endometrial cancer, gallbladder carcinoma, gastric cancer, head and neck cancer, lung cancer (e.g., small cell lung cancer or non-small cell lung cancer, or adenocarcinoma of the lung), neuroblastoma, neuroendocrine cancer, ovarian cancer, pancreatic cancer, prostate cancer, renal cancer, testicular cancer. In some embodiments, the carcinoma is bladder cancer. In some embodiments, the carcinoma is pancreatic cancer. In some embodiments, the carcinoma is breast cancer. In some embodiments, the carcinoma is head and neck cancer. In some embodiments, the carcinoma is liver cancer. In some embodiments, the carcinoma is lung cancer. In some embodiments, the carcinoma is a brain cancer. In some embodiments, the carcinoma is neuroblastoma. In some embodiments, the carcinoma is melanoma.

In some embodiments, the tumor is a liquid tumor.

In some embodiments, the step of administering results in inhibition of cell proliferation in the core of the tumor. In some embodiments, the step of administering results in slowing or inhibiting progression of the tumor. In some embodiments, the step of administering results in regression of the tumor. In some embodiments, the step of administering results in complete regression of the tumor. In some embodiments, the step of administering prevents or inhibits metastasis of tumor cells.

In some embodiments, the A-L- is a metal complex of a compound selected from the group consisting of

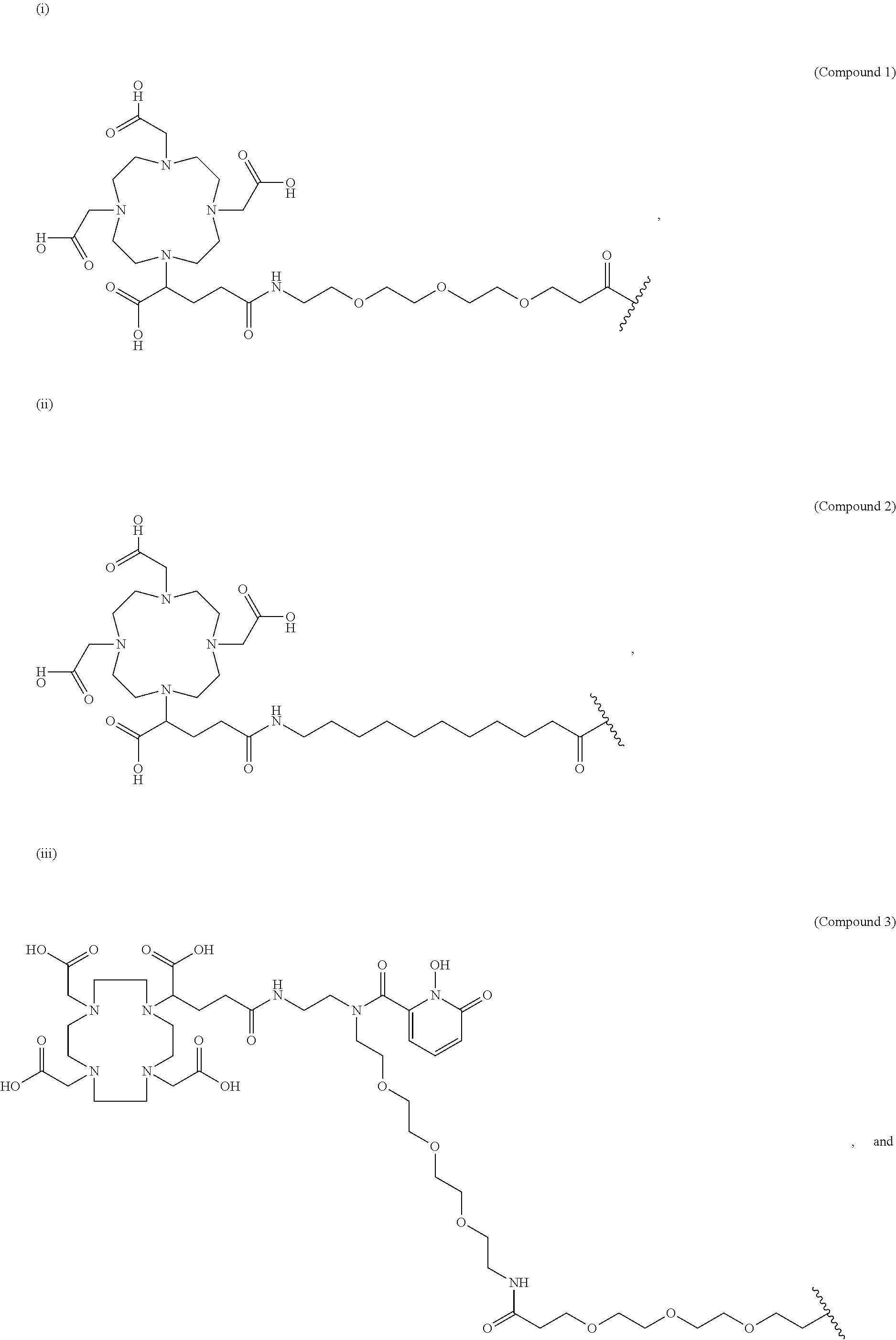 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O]

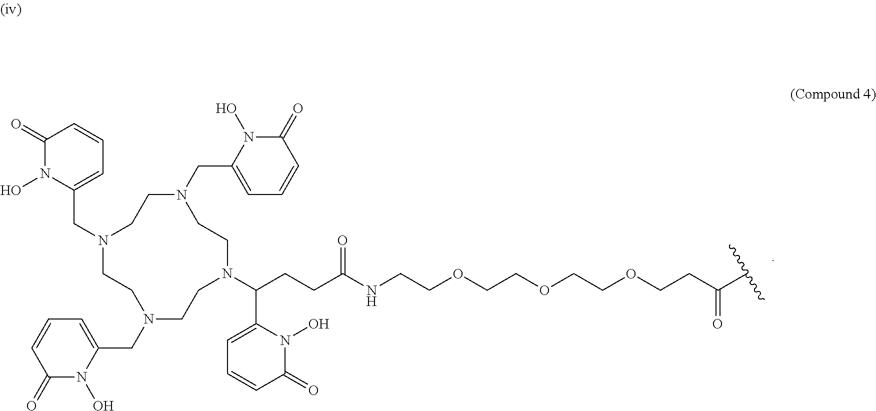 [SMILES: *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

In some embodiments, the L has the structure -L¹-(L²)ₙ-, as shown within Formula I-b:

A-L¹-(L²)ₙ-B Formula I-b

wherein
A is a metal complex of chelating moiety, wherein the metal complex comprises a Actinium-225 (²²⁵Ac) or a progeny thereof;
B is a targeting moiety;
L¹ is optionally substituted C₁-C₆ alkyl, optionally substituted C₁-C₆ heteroalkyl, or optionally substituted aryl or heteroaryl;
n is 1-5; and
each L², independently, has the structure:

(—X¹-L³-Z¹—) Formula III

wherein
X¹ is C═O(NR¹), C═S(NR¹), OC═O(NR¹), NR¹C═O(O), NR¹C═O(NR¹), —CH₂PhC═O(NR¹), —CH₂Ph(NH)C═S(NR¹), O, or NR¹; and each R¹ independently is H, optionally substituted C₁-C₆ alkyl, optionally substituted C₁-C₆ heteroalkyl, or optionally substituted aryl or heteroaryl, in which C₁-C₆ alkyl can be substituted by oxo (═O), heteroaryl, or a combination thereof;
L³ is optionally substituted C₁-C₅₀alkyl or optionally substituted C₁-C₅₀ heteroalkyl; and
Z¹ is CH₂, C═O, C═S, OC═O, NR¹C═O, or NR¹, wherein R¹ is a hydrogen or optionally substituted C₁-C₆ alkyl or pyrrolidine-2,5-dione.

In some embodiments, the radioimmunoconjugate comprises the following structure:

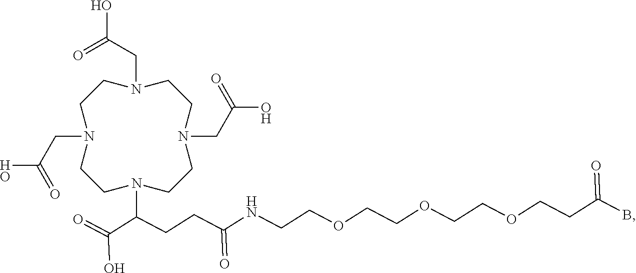

wherein B is the targeting moiety.

In some embodiments, the targeting moiety comprises a polypeptide.

In some embodiments, the targeting moiety comprises an antibody or an antigen-binding fragment thereof.

In some embodiments, the targeting moiety has a molecular weight of at least 100 kDa, at least 125 kDa, or at least 150 kDa.

In some embodiments, the targeting moiety is a small molecule.

In some embodiments, the first tumor-associated antigen is selected from the group consisting of Insulin-like Growth Factor 1 Receptor (IGF-1R), tumor epithelial marker-1 (TEM-1), and Fibroblast Growth Factor Receptor 3 (FGFR3).

In some embodiments, the subject is a mammal, e.g., a human. In some embodiments, the subject is in need of treatment or prevention of cancer. In some embodiments, the subject is diagnosed as having cancer. In some embodiments, the subject is in need of treatment of a refractory cancer.

In some embodiments, the step of administering comprises systemic administration of the radioimmunoconjugate. In some embodiments, systemic administration comprises parenteral administration, e.g., intravenous administration, intraarterial administration, intraperitoneal administration, subcutaneous administration, or intradermal administration. In some embodiments, systemic administration comprises enteric administration, e.g., trans-gastroenteric administration or oral administration.

In some embodiments, the step of administering comprises local administration of the radioimmunoconjugate. For example, the local administration may comprise peritumoral injection and/or intratumoral injection.

In some embodiments, the step of administering comprises contacting, ex vivo, the radioimmunoconjugate with a body fluid of said subject, wherein said body fluid contains at least one cancer cell.

In some embodiments, the radioimmunoconjugate is not administered in combination with another cytotoxic agent.

In some embodiments, the method further comprises administering to the subject an additional therapeutic agent after the step of administering the radioimmunoconjugate. For example, the additional therapeutic agent may be a non-cytotoxic agent. In some such embodiments, the radioimmunoconjugate is administered in a lower effective dose and/or the additional therapeutic agent is administered in a lower effective dose.

### Definitions

As used herein, “administering” an agent to a subject includes contacting a cell of said subject with the agent. In some embodiments, “administering” an agent includes contacting a cell of said subject with the agent in vivo. In some embodiments, administering an agent, for example, administering a radioimmunoconjugate, includes contacting a body fluid of a patient containing cells (for example, a cancer cell) with the agent ex vivo.

As used herein, “antibody” refers to a polypeptide whose amino acid sequence includes immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains, and may include sequence elements found in the same polypeptide chain or in different polypeptide chains.

As used herein, “antigen-binding fragment” refers to a portion of an antibody that retains the specificity of the binding characteristics of the parent antibody.

As used herein, the term “bind” or “binding,” for example, of an antibody or antigen-binding fragment thereof, means an at least temporary interaction or association with or to a target antigen. For example, “bind” or “binding” can refer to the process of a radioimmunoconjugate or a CD8⁺ T cell coming into temporary or sustained contact with a cancer cell expressing a tumor-associated antigen. In some embodiments described herein, a targeting moiety of a radioimmunoconjugate is capable of binding a tumor-associated antigen. In such embodiments, binding occurs via interaction between the tumor-associated antigen and the targeting moiety of the radioimmunoconjugate. In some embodiments described herein, a cell of a CD8⁺ T cell population is capable of binding a tumor-associated antigen. For example, binding includes the process of a CD8⁺ T cell coming into sustained contact with an antigen presenting cell via the interaction between TCR, CD8, and an MHC-bound antigen.

The terms “bifunctional chelate” or “bifunctional conjugate” are used interchangeably and, as used herein, refer to a radioimmunoconjugate compound that contains a chelating group or metal complex thereof, a linker group, and a targeting moiety (such as an antibody or antigen-binding fragment thereof that specifically binds to a tumor specific antigen or a tumor associated antigen).

The term “cancer” refers to any disease caused by the proliferation of malignant neoplastic cells, such as tumors, neoplasms, carcinomas, sarcomas, leukemias, and lymphomas.

As used herein, the term “CD8⁺ T cell population” refers to a group of one or more T cells that express the cell surface glycoprotein CD8 (cluster of differentiation 8). CD8 is a transmembrane glycoprotein that acts as a co-receptor for the T cell receptor (TCR) and binds the major histocompatibility complex. CD8 is expressed on the surface of cytotoxic T cells that mediate cancer cell destruction, in part by recognizing a specific antigen associated with the cancer cell.

The term “checkpoint inhibitor,” also known as “immune checkpoint inhibitor” (abbreviated “ICI”) refers to an agent which blocks the action of an immune checkpoint protein, e.g., blocks such immune checkpoint proteins from binding to their partner proteins. Some cancer cells are known to express immune checkpoint proteins, resulting in failure of T cells to recognize such cancer cells as targets for destruction. In general, checkpoint inhibitors facilitate destruction of cancer cells by T cells by blocking interactions between specific immune checkpoint proteins on T cells and targeted cells, where such interactions would otherwise act as a signal to inhibit targeted cell destruction by T cells. Checkpoint inhibitors include agents that block the interaction of PD-1 and PD-L1 or which block the interaction of CTLA-4 and B7-1/B7-2. Non-limiting examples of specific checkpoint inhibitors include the following antibody-based drugs: ipilimumab, nivolumab, pembrolizumab, atezolizumab, avelumab, durvalumab, and cemiplimab.

The term “chelate” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains a targeting moiety (e.g., an antibody or antigen-binding fragment thereof).

As used herein, the term “compound,” is meant to include all stereoisomers, geometric isomers, and tautomers of the structures depicted. Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis.

As used herein, the phrases “cold” or “immunologically cold,” when used in reference to a tumor or a cancer, refers to a tumor that is not responsive to checkpoint inhibition (at least in the absence of a therapeutic agent other than a checkpoint inhibitor.) Typically, an immunologically cold tumor, in the absence of a therapeutic agent, is characterized by a lack or paucity of tumor T cell infiltration. Examples of immunologically cold tumors include, without limitation, glioblastomas, ovarian cancer, prostate cancer, pancreatic cancer, and breast cancer tumors that are characterized by a lack of T cell infiltration.

As used herein, the term “core,” when used in reference to a tumor, refers to an area within the tumor that is at least about 250 μm from the margin boundary (also known as the “edge” or “border”) of the tumor.

As used herein, the phrase “in combination with,” when used in reference to therapies or therapeutic agents, refers to those situations in which a subject is simultaneously exposed to two or more therapeutic agents or modalities. In some embodiments, therapies or therapeutic agents that are administered “in combination with” one another are administered simultaneously. In some embodiments, therapies or therapeutic agents that are administered “in combination with” one another are administered sequentially. In some embodiments, therapies or therapeutic agents that are administered “in combination with” one another are administered in overlapping dosing regimens.

As used herein, the phrase “cytotoxic,” when used in reference to an agent or therapy, refers to an agent or therapy that causes direct cell killing, e.g., by directly stopping cancer cells from dividing and growing. As used herein, “cytotoxic” agents and therapies do not refer to those agents and therapies whose only contribution to cell killing is indirect, e.g., by making cells more vulnerable to killing by the immune system (such as occurs with immune checkpoint inhibition) or by inhibiting DNA damage repair.

As used herein, the phrase “detectable in a subject” means that an entity is detectable in a tissue or sample thereof (e.g., tumor sample, blood sample, etc.) in a subject.

As used herein, the terms “decrease,” “decreased,” “increase,” “increased,” “reduction,” “reduced,” and other relative terms such as “greater,” “higher,” “less” and “lower” (e.g., in reference to therapeutic outcomes or effects) have meanings relative to a reference level, as described herein.

As used herein “detection agent” refers to a molecule or atom which is useful in diagnosing a disease by locating the cells containing the antigen. Various methods of labeling polypeptides with detection agents are known in the art. Examples of detection agents include, but are not limited to, radioisotopes and radionuclides, dyes (such as with the biotin-streptavidin complex), contrast agents, luminescent agents (e.g., fluorescein isothiocyanate or FITC, rhodamine, lanthanide phosphors, cyanine, and near IR dyes), and magnetic agents, such as gadolinium chelates.

The term “DNA damage and repair inhibitor” (DDRi) refers to an agent which prevents the repair of cellular DNA damage caused by endogenous or exogenous chromosomal insults, and which acts through the inhibition of normally occurring DNA repair mechanisms and associated processes necessary for the maintenance of cellular viability.

The term “effective amount,” when used in reference to an agent (e.g., of a radioimmunoconjugate), as used herein, is that amount sufficient to effect beneficial or desired results, such as clinical results. An “effective amount” depends upon the context in which it is being applied.

The term “immunoconjugate,” as used herein, refers to a conjugate that includes a targeting moiety, such as an antibody (or antigen-binding fragment thereof), nanobody, affibody, or a consensus sequence from Fibronectin type III domain. In some embodiments, the immunoconjugate comprises an average of at least 0.10 conjugates per targeting moiety (e.g., an average of at least 0.2, 0.3, 0.4, 0.5, 0.6, 0.7, 0.8, 0.9, 1, 2, 4, 5, or 8 conjugates per targeting moiety).

As used herein, the phrase “immunogenic,” when used in reference to a tumor, refers to the tumor's ability to elicit an adaptive immune response in vivo. As used herein, a “highly immunogenic” tumor refers to a tumor that is highly responsive to immune checkpoint inhibition, e.g., exhibits tumor regression when treated with an immune checkpoint inhibitor. As used herein, a “moderately immunogenic” tumor refers to a tumor that is moderately responsive to immune checkpoint inhibition, e.g., exhibits at most delayed tumor progression but not regression in response to immune checkpoint inhibition.

As used herein, the term “infiltration,” when used in reference to cells, e.g., immune cells, refers to the movement of such cells from one tissue in a subject (e.g., the blood or the spleen) to another tissue in the subject (e.g., a tumor). Thus, the phrases “tumor infiltration” or “infiltration into a tumor” refer to movement of cells into a tumor from another location, and the phrases “tumor core infiltration” or “infiltration into the core of a tumor” refer to movement of cells into the core of a tumor.

As used herein, the phrase “lower effective dose,” when used as a term in conjunction with an agent (e.g., a therapeutic agent) refers to a dosage of the agent which is effective therapeutically in a treatment protocol at a lower dose than has previously been determined to be therapeutically effective when the agent is used as a monotherapy in reference experiments or by virtue of other therapeutic guidance.

As used herein, the phrase “margin area,” when used in reference to a tumor, refers to an area that is within about 250 μm from either side of the margin boundary of the tumor. Thus, the “margin area” as used herein includes both a 250 μm-wide area within the tumor and a 250 μm-wide area outside the tumor.

As used herein, the term “neoantigen” refers to a newly formed antigen that has not been previously recognized by the immune system. Neoantigens may arise in a number of ways, for example, from altered tumor or proteins (e.g., arising from mutations), from viral proteins, etc.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts of the compounds described here that are, within the scope of sound medical judgment, suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

Compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The terms “polypeptide” and “peptide” are used interchangeably and, as used herein, refer to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

As used herein, the phrase “preferential killing” or “preferentially kill” refers to the ability of an entity (e.g. a CD8+ T cell or an agent) to kill cells of one type at levels greater than another type, e.g., to kill tumor cells over normal cells and/or to kill cells expressing an antigen over cells not expressing the antigen. In some embodiments, the entity preferentially kills cells of one type at levels at least 2-fold, at least 3-fold, at least 4-fold, at least 5-fold, at least 6-fold, at least 7-fold, at least 8-fold, at least 9-fold, or at least 10-fold greater than another type.

As used herein, the phrase “production of a CD8⁺ T cell population” refers to the process of producing and selecting cytotoxic T cells that express CD8 and undergo V(D)J recombination and gene rearrangement of TCR DNA to produce a TCR that recognizes a specific antigen, for example, a cell surface antigen, for example, a tumor-associated antigen. Production of a CD8⁺ T cell population can also include the step of proliferation of cells of the CD8⁺ T cell population.

Production of a CD8⁺ T cell population can be followed by activation of the CD8⁺ T cell population. As used herein, “activation of a CD8⁺ T cell population” refers to the process by which cells of a CD8 T cell population are activated to bind a tumor-associated antigen and destroy a cancer cell. CD8+ T cell activation can include interaction with antigen-presenting cells, for example, matured dendritic cells. In some embodiments, methods described herein can include administering a radioimmunoconjugate, for example, to a patient in need of treatment, wherein the administering results in the activation of a CD8⁺ T cell population.

The term “radioconjugate,” as used herein, refers to any conjugate that includes a radioisotope or radionuclide, such as any of the radioisotopes or radionuclides described herein.

The term “radioimmunoconjugate,” as used herein, refers to any immunoconjugate that includes a radioisotope or radionuclide, such as any of the radioisotopes or radionuclides described herein.

The term “radioimmunotherapy,” or “radioconjugate immunotherapy” are used interchangeable. As used herein, these terms refer to a method of using a radioimmunoconjugate to produce a therapeutic effect. In some embodiments, radioimmunotherapy may include administration of a radioimmunoconjugate to a subject in need thereof, wherein administration of the radioimmunoconjugate produces a therapeutic effect in the subject. In some embodiments, radioimmunotherapy may include administration of a radioimmunoconjugate to a cell or a body fluid of a patient that includes a cell, wherein administration of the radioimmunoconjugate kills the cell. Wherein radioimmunotherapy involves the selective killing of a cell, in some embodiments the cell is a cancer cell in a subject having cancer.

As used herein, the term “radionuclide,” refers to an atom capable of undergoing radioactive decay (e.g., ³H, ¹⁴C, ¹⁵N, ¹⁸F, ³⁵S, ⁴⁷Sc, ⁵⁵Co, ⁶⁰Cu, ⁶¹Cu, ⁶²Cu, ⁶⁴Cu, ⁶⁷Cu, ⁷⁵Br, ⁷⁶Br, ⁷⁷Br, ⁸⁹Zr, ⁸⁶Y, ⁸⁷Y, ⁹⁰Y, ⁹⁷Ru, ⁹⁹Tc, ^{99m}Tc ¹⁰⁵Rh, ¹⁰⁹Pd, ¹¹¹In, ¹²³I, ¹²⁴I, ¹²⁵I, ¹³¹, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰³Pb, ²¹¹At, ²¹²Pb, ²¹²Bi, ²¹³Bi, ²²³Ra, ²²⁵Ac, ²²⁷Th, ²²⁹Th, ⁶⁶Ga, ⁶⁷Ga, ⁶⁸Ga, ⁸²Rb, ^{117m}Sn, ²⁰¹Tl). The terms radioactive nuclide, radioisotope, or radioactive isotope may also be used to describe a radionuclide. Radionuclides may be used as detection agents, as described above. In some embodiments, the radionuclide is an alpha-emitting radionuclide.

As used herein, a “reference level” refers to the level observed under appropriate reference conditions. For example, in some embodiments, the reference level is a level as determined by the use of said method with a control in an experimental animal model or clinical trial. In some embodiments, the reference level is a level in the same subject before or at the beginning of treatment. In some embodiments, the reference level is the average level in a population not being treated by said method of treatment.

As used herein, the phrase “refractory cancer” refers to a form of cancer that is unresponsive or which may be unresponsive to treatment with a currently used anti-cancer agent or current anti-cancer regimen. The term “refractory cancer” includes those cancers that are resistant to treatment at the start of treatment as well as those cancers that initially demonstrate responsiveness to treatment with an anti-cancer agent and later become unresponsive to treatment. For example, a refractory cancer can include a form of cancer where cancer cells fail to stop proliferating in response to treatment or which initially stop proliferating in response to treatment but re-commence proliferating despite further treatment with an anti-cancer agent. Apparent regression with a high frequency of recurrence may also be considered refractory. A refractory cancer may be unresponsive to a specific anti-cancer treatment with first-line, second-line, or even third-line current treatments. A patient suffering from a refractory cancer may be referred to herein as a “refractory cancer patient.”

As used herein, the phrase “specific for,” when used in the context of a T-cell receptor (TCR) being specific for an antigen, refers to the ability of the TCR recognize a peptide processed from the antigen, when that peptide is displayed by an antigen-presenting cell, e.g., when the peptide is displayed by an major histocompatibility complex (MHC)/β-2 microglobulin (β2M) complex.

As used herein, the terms “subject” and “patient” may be used interchangeable to refer to a human or non-human animal (e.g., a mammal). In some embodiments described herein, a patient is in need of treatment of a refractory cancer. Such a patient may also be referred to as a “refractory cancer patient.”

By “substantial identity” or “substantially identical” is meant a polypeptide sequence that has the same polypeptide sequence, respectively, as a reference sequence, or has a specified percentage of amino acid residues, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

The term “targeting moiety” as used herein refers to any molecule or any part of a molecule that binds to a given target. In some embodiments, the targeting moiety is a small molecule, a protein or polypeptide such as an antibody or antigen binding fragment thereof, a nanobody, an affibody, or a consensus sequence from a Fibronectin type III domain.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the term “tumor-associated antigen” or “tumor associated antigen” means an antigen that is present on tumor cells at a significantly greater amount than on normal cells.

Tumors:

As used herein, a “primary tumor” refers to an original tumor growth at a primary site of origin and is not the product of metastasis.

As used herein, a “secondary tumor” includes a tumor growth that has spread from a primary site of origin to a secondary anatomical site, often through the process of metastasis. In the context of an experimental animal model, a “secondary tumor” can also refer to a tumor that forms in a tumor re-challenge experiment, in which the animal is again challenged with the same type of cancer cells as the animal had previously received.

A “solid tumor” a cancer comprising an abnormal mass of tissue, e.g., sarcomas, carcinomas, and lymphomas.

A “liquid tumor” as used herein, is a cancer present in a body fluid, e.g., lymphomas and leukemias.

As used herein, the term “tumor-specific antigen” or “tumor specific antigen” refers to an antigen that is endogenously present only on tumor cells.

### BRIEF DESCRIPTION OF THE DRAWINGS

FIG. 1A illustrates an experiment described in Example 2 and conducted using a moderately immunogenic syngeneic mouse model, a CT-26 mouse colon cancer model.

FIG. 1B shows tumor growth curves for mice treated with vehicle, TAB-199 (a human monoclonal IGF-1R antibody), or [²²⁵Ac]-FPI-1792 (‘TAT’) at 100 nCi or 200 nCi. [²²⁵Ac]-FPI-1792 is a radioimmunoconjugate comprising TAB-199 conjugated to a DOTA chelating moiety via a Fast-Clear™ linker, with the ²²⁵Ac complexed to the DOTA moiety.

FIG. 1C is a series of panels showing representative Ki67-stained CT-26 tumor tissues from mice used in experiments described in Example 2. Ki67 is a marker of cell proliferation. Tissue sections were obtained from tumors dissected 7 days after administration of vehicle control (D7-Control-untreated) or 200 nCi of [²²⁵Ac]-FPI-1792 (D7-200 nCi Ac-TAB-199). Upper panels, 2× magnification. Lower panels, 20× magnification.

FIG. 1D is a series of panels showing representative CD8-stained CT-26 tumor tissues from mice used in experiments described in Example 2. CD8 is a marker of T cells. Tissue sections were obtained from tumors dissected 7 days after administration of vehicle control (D7-Control-untreated) or 200 nCi of [²²⁵Ac]-FPI-1792 (D7-200 nCi Ac-TAB-199). Upper panels, 2× magnification. Lower panels, 20× magnification.

FIG. 1E is a series of panels showing representative Granzyme B-stained CT-26 tumor tissues from mice used in experiments described in Example 2. Granzyme B is a serine protease found in the granules of natural killer cells and cytotoxic T cells. Tissue sections were obtained from tumors dissected 7 days after administration of vehicle control (D7-Control-untreated) or 200 nCi of [²²⁵Ac]-FPI-1792 (D7-200nCi Ac-TAB-199). Upper panels, 2× magnification. Lower panels, 20× magnification.

FIG. 2A illustrates an experiment described in Example 3 and conducted using a moderately immunogenic syngeneic mouse model, a CT-26 mouse colon cancer model.

FIG. 2B shows a dosing schedule for experiments described in Example 3. Numbers at the top of FIG. 3B indicate days.

FIG. 2C shows tumor growth curves for mice treated with vehicle, TAB-199 (a human monoclonal IGF-1R antibody); 200 nCi [²²⁵Ac]-FPI-1792 (‘TAT’); a combination of 200 nCi [²²⁵Ac]-FPI-1792 and a PD-1 antibody; a combination of 200 nCi [²²⁵Ac]-FPI-1792 and a CTLA-4 antibody; and a combination of 200 nCi [²²⁵Ac]-FPI-1792, a PD-1 antibody, and a CTLA-4 antibody. (See Example 3.)

FIG. 2D is a series of panels showing representative Ki67-stained CT-26 tumor tissues from mice used in experiments described in Example 3. Tissue sections were obtained from tumors dissected 12 days after administration of vehicle control, TAB-199, [²²⁵Ac]-FPI-1792, [²²⁵Ac]-FPI-1792+anti-CTLA-4, [²²⁵Ac]-FPI-1792+anti-PD-1, or [²²⁵Ac]-FPI-1792+anti-CTLA-4+anti-PD-1.

FIG. 2E is a series of panels showing representative CD8-stained CT-26 tumor tissues from mice used in experiments described in Example 3. Tissue sections were obtained from tumors dissected 12 days after administration of vehicle control, TAB-199, [²²⁵Ac]-FPI-1792, [²²⁵Ac]-FPI-1792+anti-CTLA-4, [²²⁵Ac]-FPI-1792+anti-PD-1, or [²²⁵Ac]-FPI-1792+anti-CTLA-4+anti-PD-1.

FIG. 2F is a series of panels showing representative Granzyme B-stained CT-26 tumor tissues from mice used in experiments described in Example 3. Tissue sections were obtained from tumors dissected 12 days after administration of vehicle control, TAB-199, [²²⁵Ac]-FPI-1792, [²²⁵Ac]-FPI-1792+anti-CTLA-4, [²²⁵Ac]-FPI-1792+anti-PD-1, or [²²⁵Ac]-FPI-1792+anti-CTLA-4+anti-PD-1.

FIG. 2G is a graph depicting quantification of Ki67-positive cells in tumor tissues from experiments described in Example 3. Counts of total cell nuclei and of Ki67-positive cells were performed on five different areas from tumor core on tissue sections obtained from tumors dissected 12 days after administration of vehicle control, TAB-199, [²²⁵Ac]-FPI-1792, [²²⁵Ac]-FPI-1792+anti-CTLA-4, [²²⁵Ac]-FPI-1792+anti-PD-1, or [²²⁵Ac]-FPI-1792+anti-CTLA-4+anti-PD-1. p-values: *p<0.05, **p<0.01, ***p<0.001.

FIG. 2H is a graph depicting quantification of CD8-positive cells in tumor tissues from experiments described in Example 3. Counts of total cell nuclei and of CD8-positive cells were performed on five different areas from tumor core on tissue sections obtained from tumors dissected 12 days after administration of vehicle control, TAB-199, [²²⁵Ac]-FPI-1792, [²²⁵Ac]-FPI-1792+anti-CTLA-4, [²²⁵Ac]-FPI-1792+anti-PD-1, or [²²⁵Ac]-FPI-1792+anti-CTLA-4+anti-PD-1. *p<0.05, **p<0.01, ***p<0.001.

FIG. 2I is a graph depicting quantification of Granzyme B-positive cells in tumor tissues from experiments described in Example 3. Counts of total cell nuclei and of Granzyme B-positive cells were performed on five different areas from tumor core on tissue sections obtained from tumors dissected 12 days after administration of vehicle control, TAB-199, [²²⁵Ac]-FPI-1792, [²²⁵Ac]-FPI-1792+anti-CTLA-4, [²²⁵Ac]-FPI-1792+anti-PD-1, or [²²⁵Ac]-FPI-1792+anti-CTLA-4+anti-PD-1. *p<0.05, **p<0.01, ***p<0.001.

FIG. 3A illustrates a tumor re-challenge experiment described in Example 4 and conducted using a moderately immunogenic syngeneic mouse model, a CT-26 mouse colon cancer model.

FIG. 3B is a set of panels showing CD8 immunostaining of implanted CT-26 allograft tumor tissue 11 days after a second round of CT-26 allograft tumor cell implantation in mice that were untreated (control tumor) or in mice originally administered 200 nCi of radioimmunoconjugate at the start of the experiment (secondary tumor), as described in Example 4.

FIG. 3C shows secondary tumor growth curves for mice treated with vehicle, [²²⁵Ac]-FPI-1792 (‘TAT’), [²²⁵Ac]-FPI-1792+anti-PD1, [²²⁵Ac]-FPI-1792+CTLA4, or [²²⁵Ac]-FPI-1792+anti-PD1+anti-CTLA4. Tumor volume is plotted against number of days after tumor re-challenge. Experiments are described in Example 4.

FIG. 4A illustrates a tumor re-challenge experiment conducted on mice using a moderately immunogenic syngeneic mouse model, a CT-26 mouse colon cancer model. Example 5 describes analyses of T-cells in mice subjected to the depicted re-challenge experiment.

FIG. 4B depicts frequencies of CD8+ T cells in spleen and tumors from mice that were untreated, or treated with [²²⁵Ac]-FPI-1792+anti-PD-1, [²²⁵Ac]-FPI-1792+anti-CTLA-4, or [²²⁵Ac]-FPI-1792+anti-CTLA-4+anti-PD-1, as described in Example 5.

FIG. 4C is a schematic depicting the tetramer analysis used in experiments described in Example 5. Tetramer analysis allows enumeration of antigen-specific CD8+ T cells when the MHC class I molecule and peptide sequence of the antigen is known. The peptide antigen used in this analysis was AH1 (SPSYVYHGF (SEQ ID NO: 1)), an immunodominant CD8+ T cell epitope from CT-26 (the colon cancer cell line used in the syngeneic mouse model from which the tumors were generated).

FIG. 4D depicts frequencies of (CT-26) antigen-specific CD8+ T cells (as percentages of all CD8+ T cells) in spleen and tumors from mice that were untreated, or treated with [²²⁵Ac]-FPI-1792+anti-PD-1, [²²⁵Ac]-FPI-1792+anti-CTLA-4, or [²²⁵Ac]-FPI-1792+anti-CTLA-4+anti-PD-1, as described in Example 5. Frequencies of CD8+ T cells specific for a CT26 peptide (AH1) were determined using tetramer analysis. (See FIG. 4C.)

FIG. 5A is an illustration of an experiment described in Example 6 and conducted using an immunogenically cold syngeneic mouse model, a 4T1 triple negative breast cancer model.

FIG. 5B shows 4T1 tumor growth curves for mice treated with vehicle, a CTLA-4 antibody, [²²⁵Ac]-FPI-1792 (‘TAT’), or a combination of both a CTLA-4 antibody and [²²⁵Ac]-FPI-1792, as described in Example 6.

FIG. 5C shows 4T1 tumor growth curves for mice treated with vehicle, a RMP1-14 (a PD-1 antibody), [²²⁵Ac]-FPI-1792 (‘TAT’), or a combination of both a RMP1-14 and [²²⁵Ac]-FPI-1792, as described in Example 6.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Described herein are methods of inducing CD8+ T cell infiltration into a tumor (e.g., into the core of a tumor) in a patient in need thereof. Disclosed methods comprise a step of administering to the patient a radioimmunoconjugate having a structure as described further herein, or a pharmaceutical composition thereof.

### Radioimmunoconjugates

Radioimmunoconjugates used in accordance with the disclosure generally comprise the structure of Formula I-a:

A-L-B Formula I-a

wherein
A is a metal complex of chelating moiety, wherein the metal complex comprises Actinium-225 (²²⁵Ac) or a progeny thereof,
L is a linker, and
B is a targeting moiety capable of binding a first tumor-associated antigen, with the proviso that if A-L- is a metal complex of Compound 1 as shown below, then B is not AVE1642.

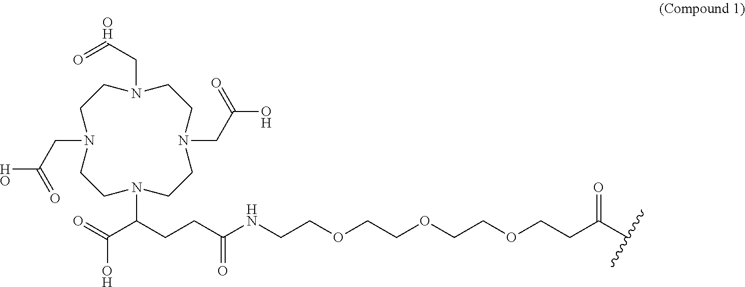

In some embodiments, A-L- is a metal complex of a compound selected from the group consisting of

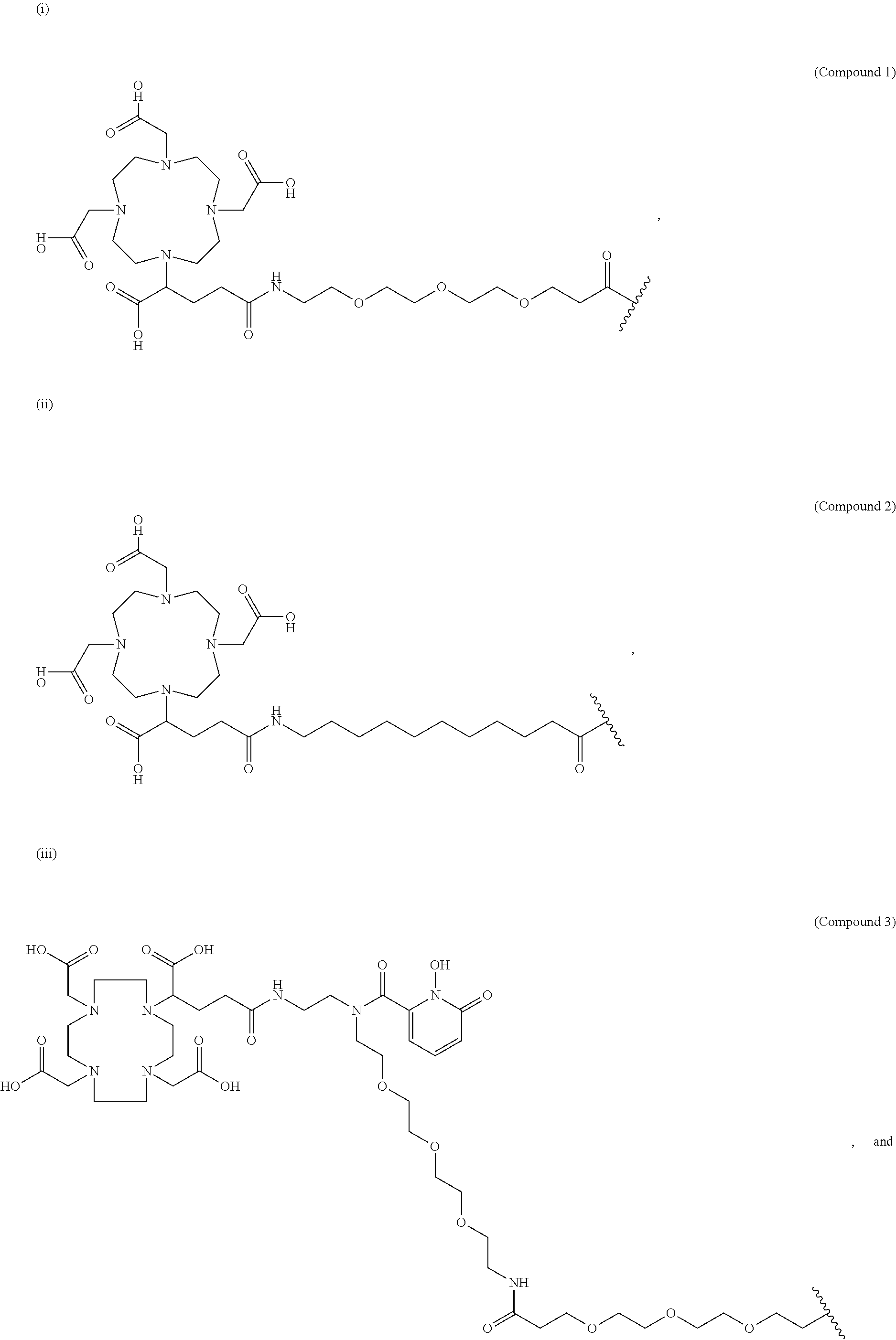 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O]

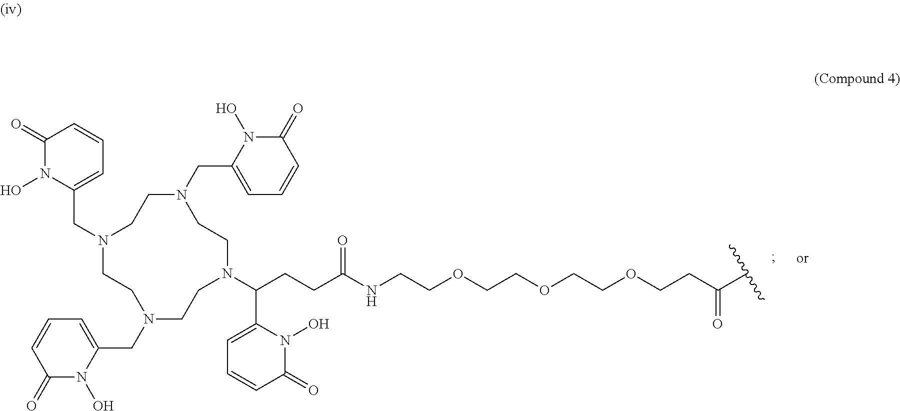 [SMILES: *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

In some embodiments, the radioimmunoconjugate has or comprises the structure shown in Formula II:

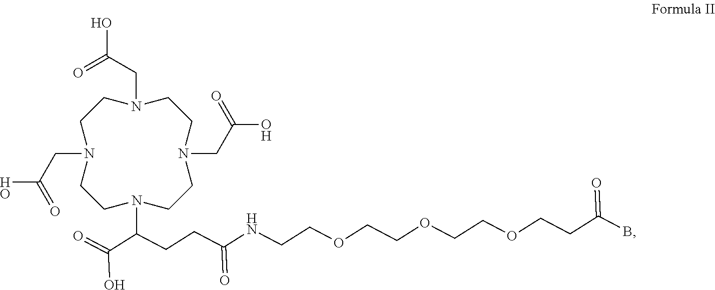

wherein B is the targeting moiety and a ²²⁵Ac is complexed to the 1,4,7,10-tetraazacyclododecane-1,4,7,10-tetraacetic acid (DOTA) moiety.

In some embodiments, the average ratio or median ratio of the chelating moiety to the targeting moiety is eight or less, seven or less, six or less, five or less, four or less, three or less, two or less, or about one. In some radioimmunoconjugates, the average ratio or median ratio of the chelating moiety to the targeting moiety is about one.

In some embodiments, after the radioimmunoconjugate is administered to a mammal, the proportion of radiation (of the total amount of radiation that is administered) that is excreted by the intestinal route, the renal route, or both is greater than the proportion of radiation excreted by a comparable mammal that has been administered a reference radioimmunoconjugate. By “reference immunoconjugate” it is meant a known radioimmunoconjugate that differs from a radioimmunoconjugate described herein at least by (1) having a different linker; (2) having a targeting moiety of a different size and/or (3) lacking a targeting moiety. In some embodiments, the reference radioimmunoconjugate is selected from the group consisting of [⁹⁰Y]-ibritumomab tiuxetan (Zevalin (90Y)) and [¹¹¹In]-ibritumomab tiuxetan (Zevalin (In-111)).

In some embodiments, the proportion of radiation excreted by a given route or set of routes) is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater than the proportion of radiation excreted by the same route(s) by a comparable mammal that has been administered a reference radioimmunoconjugate. In some embodiments, the proportion of radiation excreted is at least 1.5-fold, at least 2-fold, at least 2.5-fold, at least 3-fold, at least 3.5 fold, at least 4-fold, at least 4.5 fold, at least 5 fold, at least 6-fold, at least 7-fold, at least 8-fold, at least 9-fold, or at least 10-fold greater than proportion of radiation excreted by a comparable mammal that has been administered a reference radioimmunoconjugate. The extent of excretion can be measured by methods known in the art, e.g., by measuring radioactivity in urine and/or feces and/or by measuring total body radioactivity over a period time. See also, e.g., International Patent Publication WO 2018/024869.

In some embodiments, the extent of excretion is measured at a time period of at least or about 12 hours after administration, at least or about 24 hours after administration, at least or about 2 days after administration, at least or about 3 days after administration, at least or about 4 days after administration, at least or about 5 days after administration, at least or about 6 days after administration, or at least or about 7 days, after administration.

In some embodiments, when radioimmunoconjugates according to the present disclosure are administered to a mouse, (a) less than 15% of total radiation administered is excreted by day 1 after administration, and (b) at least 15% of total radiation administered is excreted by day 7 after administration.

In some embodiments, when radioimmunoconjugates according to the present disclosure are administered to a mouse, less than 10% of total radiation administered is excreted by the renal route by day 2 after administration.

In some embodiments, when radioimmunoconjugates according to the present disclosure are administered to a mouse, (a) less than 15% of total radiation administered is excreted by day 1 after administration; (b) less than 10% of total radiation administered is excreted by the renal route by day 2 after administration; and (c) at least 15% of total radiation administered is excreted by day 7 after administration.

In some embodiments in which the targeting moiety is a small molecule and/or has a molecular weight of less than 50 kDa, when the radioimmunoconjugate is administered to a mouse, (a) less than 15% of total radiation administered is excreted by day 1 after administration, and (b) at least 15% of total radiation administered is excreted by day 7 after administration.

In some embodiments in which the targeting moiety is a small molecule and/or has a molecular weight of less than 50 kDa, when the radioimmunoconjugate is administered to a mouse, (a) less than 15% of total radiation administered is excreted by day 1 after administration; (b) less than 10% of total radiation administered is excreted by the renal route by day 2 after administration; and (b) at least 15% of total radiation administered is excreted by day 7 after administration.

In some embodiments, after a radioimmunoconjugate has been administered to a mammal, the radioimmunoconjugate exhibits decreased off-target binding effects (e.g., toxicities) as compared to a reference conjugate (e.g., a reference immunoconjugate such as a reference radioimmunoconjugate). In some embodiments, this decreased off-target binding effect is a feature of a radioimmunoconjugate that also exhibits a greater excretion rate as described herein.

### Chelating Moieties

Examples of suitable chelating moieties include, but are not limited to, DOTA (1,4,7,10-tetraazacyclododecane-1,4,7,10-tetraacetic acid), DOTMA (1R,4R,7R,10R)-α, α′, α″, α′″-tetramethyl-1,4,7,10-tetraazacyclododecane-1,4,7,10-tetraacetic acid, DOTAM (1,4,7,10-tetrakis(carbamoylmethyl)-1,4,7,10-tetraazacyclododecane), DOTPA (1,4,7,10-tetraazacyclododecane-1,4,7,10-tetra propionic acid), DO3AM-acetic acid (2-(4,7,10-tris(2-amino-2-oxoethyl)-1,4,7,10-tetraazacyclododecan-1-yl)acetic acid), DOTA-GA anhydride (2,2′,2″-(10-(2,6-dioxotetrahydro-2H-pyran-3-yl)-1,4,7,10-tetraazacyclododecane-1,4,7-triyl)triacetic acid, DOTP (1,4,7,10-tetraazacyclododecane-1,4,7,10-tetra(methylene phosphonic acid)), DOTMP (1,4,6,10-tetraazacyclodecane-1,4,7,10-tetramethylene phosphonic acid, DOTA-4AMP (1,4,7,10-tetraazacyclododecane-1,4,7,10-tetrakis(acetamido-methylenephosphonic acid), CB-TE2A (1,4,8,11-tetraazabicyclo[6.6.2]hexadecane-4,11-diacetic acid), NOTA (1,4,7-triazacyclononane-1,4,7-triacetic acid), NOTP (1,4,7-triazacyclononane-1,4,7-tri(methylene phosphonic acid), TETPA (1,4,8,11-tetraazacyclotetradecane-1,4,8,11-tetrapropionic acid), TETA (1,4,8,11-tetraazacyclotetradecane-1,4,8,11-tetra acetic acid), HEHA (1,4,7,10,13,16-hexaazacyclohexadecane-1,4,7,10,13,16-hexaacetic acid), PEPA (1,4,7,10,13-pentaazacyclopentadecane-N,N′,N″,N′″, N″″-pentaacetic acid), H₄octapa (N,N′-bis(6-carboxy-2-pyridylmethyl)-ethylenediamine-N,N′-diacetic acid), H₂dedpa (1,2-[[6-(carboxy)-pyridin-2-yl]-methylamino]ethane), H₆phospa (N,N′-(methylenephosphonate)-N,N′-[6-(methoxycarbonyl)pyridin-2-yl]-methyl-1,2-diaminoethane), TTHA (triethylenetetramine-N,N,N′,N″,N′″, N′″-hexaacetic acid), DO2P (tetraazacyclododecane dimethanephosphonic acid), HP-DO3A (hydroxypropyltetraazacyclododecanetriacetic acid), EDTA (ethylenediaminetetraacetic acid), Deferoxamine, DTPA (diethylenetriaminepentaacetic acid), DTPA-BMA (diethylenetriaminepentaacetic acid-bismethylamide), HOPO (octadentate hydroxypyridinones), or porphyrins.

### Linkers

In some embodiments, the linker is as shown within the structure of Formula I-b, as that part of Formula I-b absent A and B:

A-L¹-(L²)ₙ-B Formula I-b

(A and B are as defined in Formula I-a).

Thus, in some embodiments, the linker is -L¹-(L²)ₙ-, wherein:
L¹ is optionally substituted C₁-C₆ alkyl, optionally substituted C₁-C₆ heteroalkyl, or optionally substituted aryl or heteroaryl;
n is 1-5; and
each L², independently, has the structure:

(—X¹-L³-Z¹—) Formula III
wherein X¹ is C═O(NR¹), C═S(NR¹), OC═O(NR¹), NR¹C═O(O), NR¹C═O(NR¹), —CH₂PhC═O(NR¹), —CH₂Ph(NH)C═S(NR¹), O, or NR¹; and each R¹ independently is H, optionally substituted C₁-C₆ alkyl, optionally substituted C₁-C₆ heteroalkyl, or optionally substituted aryl or heteroaryl, in which C₁-C₆ alkyl can be substituted by oxo (═O), heteroaryl, or a combination thereof;
L³ is optionally substituted C₁-C₅₀ alkyl or optionally substituted C₁-C₅₀ heteroalkyl (e.g., C₅-C₂₀ polyethylene glycol);
Z¹ is CH₂, C═O, C═S, OC═O, NR¹C═O, or NR¹, wherein R¹ is a hydrogen or optionally substituted C₁-C₆ alkyl or pyrrolidine-2,5-dione.

In some embodiments, L¹ is substituted C₁-C₆ alkyl or substituted C₁-C₆ heteroalkyl, the substituent comprising a heteroaryl group (e.g., six-membered nitrogen-containing heteroaryl).

In some embodiments, L³ is substituted C₁-C₅₀ alkyl or substituted C₁-C₅₀ heteroalkyl, the substituent comprising a heteroaryl group (e.g., six-membered nitrogen-containing heteroaryl).

In some embodiments, A is a macrocyclic chelating moiety comprising one or more heteroaryl groups (e.g., six-membered nitrogen-containing heteroaryl).

### Cross-Linking Groups

In some embodiments, radioimmunoconjugates comprise a cross-linking group instead of or in addition to the targeting moiety (e.g., B in Formula I comprises a cross-linking group).

A cross-linking group is a reactive group that is able to join two or more molecules by a covalent bond. Cross-linking groups may be used to attach the linker and chelating moiety to a therapeutic or targeting moiety. Cross-linking groups may also be used to attach the linker and chelating moiety to a target in vivo. In some embodiments, the cross-linking group is an amino-reactive, methionine reactive or thiol-reactive cross-linking group, or a sortase-mediated coupling. In some embodiments, the amino-reactive or thiol-reactive cross-linking group comprises an activated ester such as a hydroxysuccinimide ester, 2,3,5,6-tetrafluorophenol ester, 4-nitrophenol ester or an imidate, anhydride, thiol, disulfide, maleimide, azide, alkyne, strained alkyne, strained alkene, halogen, sulfonate, haloacetyl, amine, hydrazide, diazirine, phosphine, tetrazine, isothiocyanate, or oxaziridine. In some embodiments, the sortase recognition sequence may comprise of a terminal glycine-glycine-glycine (GGG) and/or LPTXG amino acid sequence, where X is any amino acid. A person having ordinary skill in the art will understand that the use of cross-linking groups is not limited to the specific constructs disclosed herein, but rather may include other known cross-linking groups.

### Targeting Moieties

Targeting moieties include any molecule or any part of a molecule that is capable of binding to a given target, e.g., a tumor-associated antigen or a tumor-specific antigen. In some embodiments, the targeting moiety is capable of binding to an epitope within the target. In the context of a method comprising administering a radioimmunoconjugate to a patient having a tumor, the target may be, e.g., an antigen (e.g., a tumor-associated antigen or a tumor-specific antigen) expressed by at least some cells in the tumor.

In some embodiments, the targeting moiety is capable of binding to an antigen (e.g., a tumor-associated antigen or tumor-specific antigen) expressed by at least some cells in the tumor. Examples of suitable tumor-associated antigens include, but are not limited to, IGF-1R, tumor endothelial marker-1 (TEM-1, also known as endosialin), and FGFR3. In embodiments in which the tumor-associated antigen is IGF-1R, the targeting moiety is not AVE1642 (a humanized monoclonal IGF-1R antibody (Sanofi®-Aventis/Immunogen)).

In some embodiments, the targeting moiety has a molecular weight of at least 50 kDa, at least 75 kDa, at least 100 kDa, at least 125 kDa, at least 150 kDa, at least 175 kDa, at least 200 kDa, at least 225 kDa, at least 250 kDa, at least 275 kDa, or at least 300 kDa. In some embodiments, the targeting moiety has a molecular weight of at least 100 kDa, at least 125 kDa, or at least 150 kDa.

In some embodiments, the targeting moiety comprises a small molecule. For example, small molecules that are targeting ligands (e.g. high affinity targeting ligands), or derivatives thereof, may be used as a targeting moiety. Examples of suitable small molecules include, without limitation, PSMA-617 (a prostate-specific membrane antigen ligand) and 3BP-227 (a neurotensin receptor type 1 antagonist).

In some embodiments, a moiety is both a targeting and a therapeutic moiety, i.e., the moiety is capable of binding to a given target and also confers a therapeutic benefit.

In some embodiments, the targeting moiety comprises a protein or polypeptide (e.g., a modified polypeptide). In some embodiments, the targeting moiety is selected from the group consisting of antibodies or antigen-binding fragments thereof, avimers, nanobodies, affibodies, and consensus sequences from Fibronectin type III domains (e.g., Centyrins or adnectins), or molecules comprising any of the foregoing.

### Antibodies

In some embodiments, the targeting moiety comprises an antibody or antigen-binding fragment thereof. Antibodies typically comprise two identical light polypeptide chains and two identical heavy polypeptide chains linked together by disulfide bonds. The first domain located at the amino terminus of each chain is variable in amino acid sequence, providing the antibody-binding specificities of each individual antibody. These are known as variable heavy (VH) and variable light (VL) regions. The other domains of each chain are relatively invariant in amino acid sequence and are known as constant heavy (CH) and constant light (CL) regions. Light chains typically comprise one variable region (VL) and one constant region (CL). An IgG heavy chain includes a variable region (VH), a first constant region (CH1), a hinge region, a second constant region (CH2), and a third constant region (CH3). In IgE and IgM antibodies, the heavy chain includes an additional constant region (CH4).

Antibodies suitable for use with the present disclosure can include, for example, monoclonal antibodies, polyclonal antibodies, multi-specific antibodies, human antibodies, humanized antibodies, camelid antibodies, chimeric antibodies, single-chain Fvs (scFv), disulfide-linked Fvs (sdFv), and anti-idiotypic (anti-Id) antibodies, and antigen-binding fragments of any of the above. In some embodiments, the antibody or antigen-binding fragment thereof is humanized. In some embodiments, the antibody or antigen-binding fragment thereof is chimeric. Antibodies can be of any type (e.g., IgG, IgE, IgM, IgD, IgA and IgY), class (e.g., IgG1, IgG2, IgG3, IgG4, IgA1 and IgA2) or subclass.

The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)2 fragment, a Fd fragment, a Fv fragment, a scFv fragment, a dAb fragment (Ward et al., (1989) Nature 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

Antibodies or antigen-binding fragments described herein can be produced by any method known in the art for the synthesis of antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using the methods described in, e.g., Morrison, 1985, Science 229:1202, and humanized antibodies by methods described in, e.g., U.S. Pat. No. 6,180,370.

Additional antibodies described herein are bispecific antibodies and multivalent antibodies, as described in, e.g., Segal et al., J. Immunol. Methods 248:1-6 (2001); and Tutt et al., J. Immunol. 147: 60 (1991), or any of the molecules described herein.

In certain embodiments, amino acid sequence variants of antibodies or antigen-binding fragments thereof are contemplated; e.g., variants that retain the ability to bind to an intended target. For example, it may be desirable to improve the binding affinity and/or other biological properties of the antibody or antigen-binding fragment thereof. Amino acid sequence variants of an antibody or antigen-binding fragment thereof may be prepared by introducing appropriate modifications into the nucleotide sequence encoding the antibody or antigen-binding fragment thereof, or by peptide synthesis. Such modifications include, for example, deletions from and/or insertions into and/or substitutions of residues within the amino acid sequences of the antibody or antigen-binding fragment thereof. Any combination of deletion, insertion and substitution can be made to arrive at the final construct, provided that the final construct possesses desired characteristics, e.g. antigen binding.

In some embodiments, the antibody or antigen binding fragment thereof is an inhibitory antibody (also called “antagonistic antibody”) or antigen-binding fragment thereof, e.g., the antibody or antigen binding fragment thereof at least partially inhibits one or more functions of the target.

In certain embodiments, the antibody or antigen-binding fragment thereof has a dissociation constant (Kd) of ≤1 μM, ≤100 nM, ≤10 nM, ≤1 nM, ≤0.1 nM, ≤0.01 nM, or ≤0.001 nM. In some embodiments, the antibody or antigen-binding fragment thereof has a dissociation constant (Kd) of between 1 nM and 10 nM (inclusive of endpoints) or between 0.1 nM and 1 nM (inclusive of endpoints).

In some embodiments, Kd is measured by a radio-labeled antigen binding assay (radioimmunoassay, RIA) performed with the Fab version of an antibody or antigen-binding fragment thereof of interest and its antigen.

According to some embodiments, Kd is measured using surface plasmon resonance assays with immobilized antigen. In some embodiments, the antibodies or antigen-binding fragments thereof are human monoclonal antibodies directed against an epitope of the target (e.g., tumor-associated antigen or tumor-specific antigen).

The antibody or antigen-binding fragment thereof may be any antibody or antigen-binding fragment thereof of natural and/or synthetic origin, e.g. an antibody of mammalian origin. In some embodiments, the constant domain, if present, is a human constant domain. In some embodiments, the variable domain is a mammalian variable domain, e.g., a humanized or a human variable domain.

In some embodiments, antibodies used in accordance with this disclosure are monoclonal antibodies. In some embodiments, antibodies are recombinant murine antibodies, chimeric, humanized or fully human antibodies, multispecific antibodies (e.g., bispecific antibodies), or antigen-binding fragments thereof.

In some embodiments, are further coupled to other moieties for, e.g., drug targeting and imaging applications.

In some embodiments, e.g., for diagnostic purposes, the antibody or antigen-binding fragment thereof is labelled, i.e. coupled to a labelling group. Non-limiting examples of suitable labels include radioactive labels, fluorescent labels, suitable dye groups, enzyme labels, chromogenes, chemiluminescent groups, biotinyl groups, predetermined polypeptide epitopes recognized by a secondary reporter etc. In some embodiments, one or more labels are covalently bound to the antibody or antigen-binding fragment thereof.

Those labelled antibodies or antigen-binding fragments thereof (also referred to as “antibody conjugates”) may in particular be used in immunohistochemistry assays or for molecular imaging in vivo.

In some embodiments, e.g., for therapeutic purposes, the antibody or antigen-binding fragment thereof is further conjugated with an effector group, in particular, a therapeutic effector group such as a cytotoxic agent or a radioactive group agent.

### Polypeptides

Polypeptides include, for example, any of a variety of hematologic agents (including, for instance, erythropoietin, blood-clotting factors, etc.), interferons, colony stimulating factors, antibodies, enzymes, and hormones. The identity of a particular polypeptide is not intended to limit the present disclosure, and any polypeptide of interest can be a polypeptide in the present methods.

A polypeptide described herein can include a target-binding domain that is capable of binding to a target of interest (e.g., a tumor-associated antigen or a tumor-specific antigen). For example, a polypeptide, can be capable of binding to a transmembrane polypeptide (e.g., receptor) or ligand (e.g., a growth factor).

In some embodiments, the polypeptide is a synthetic polypeptide, e.g., an analogue of a naturally occurring polypeptide (e.g., somatostatin).

Non-limiting examples of suitable polypeptides include cyclic octapeptides such as octreotate and octreotide. For example, octreotate and octreotide may be conjugated to DOTA bifunctional chelators to form DOTA-TATE and DOTA-TOC, respectively, which are often used for high-SSTR2 expressing cancers.

### Modified Polypeptides

Polypeptides suitable for use with compositions and methods of the present disclosure may have a modified amino acid sequence. Modified polypeptides may be substantially identical to the corresponding reference polypeptide (e.g., the amino acid sequence of the modified polypeptide may have at least 50%, 60%, 70%, 75%, 80%, 85%, 90%, 95%, 96%, 97%, 98%, 99%, or 100% identity to the amino acid sequence of the reference polypeptide). In certain embodiments, the modification does not destroy significantly a desired biological activity. The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified polypeptide may have or may optimize a characteristic of a polypeptide, such as in vivo stability, bioavailability, toxicity, immunological activity, immunological identity, and conjugation properties.

Modifications include those by natural processes, such as post-translational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from post-translational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to flavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy-terminus of a polypeptide herein can facilitate conjugation of these polypeptides by, e.g., disulfide bonding. For example, a polypeptide can be modified to include a single cysteine residue at the amino-terminus or a single cysteine residue at the carboxy-terminus. Amino acid substitutions can be conservative (i.e., wherein a residue is replaced by another of the same general type or group) or non-conservative (i.e., wherein a residue is replaced by an amino acid of another type). In addition, a naturally occurring amino acid can be substituted for a non-naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, N-protected amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 1. If such substitutions result in a change not desired, then other type of substitutions, denominated “exemplary substitutions” in Table 1, or as further described herein in reference to amino acid classes, are introduced and the products screened.

TABLE 1
Amino acid substitutions

| | Exemplary | Conservative |
|---|---|---|
| Original residue | substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, | Leu |
| | norleucine | |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Substantial modifications in function or immunological identity are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, and/or (c) the bulk of the side chain.

### Pharmaceutical Compositions

In some embodiments, methods comprise administering a pharmaceutical composition of a radioimmunoconjugate as described herein. Such pharmaceutical compositions can be formulated for use in a variety of drug delivery systems. One or more physiologically acceptable excipients or carriers can also be included in a pharmaceutical composition for proper formulation. Non-limiting examples of suitable formulations compatible for use with the present disclosure include those described in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

Pharmaceutical compositions may be formulated for any of a variety of routes of administration discussed herein (see, e.g., the “Administration and Dosage” subsection herein), Sustained release administration is contemplated, by such means as depot injections or erodible implants or components. Thus, the present disclosure provides pharmaceutical compositions that include agents disclosed herein (e.g., radioimmunoconjugates) dissolved or suspended in an acceptable carrier, preferably an aqueous carrier, e.g., water, buffered water, saline, or PBS, among others. In some embodiments, pharmaceutical compositions contain pharmaceutically acceptable auxiliary substances to approximate physiological conditions, such as pH adjusting and buffering agents, tonicity adjusting agents, wetting agents, or detergents, among others. In some embodiments, pharmaceutical compositions are formulated for oral delivery and may optionally contain inert ingredients such as binders or fillers for the formulation of a unit dosage form, such as a tablet or a capsule. In some embodiments, pharmaceutical compositions are formulated for local administration and may optionally contain inert ingredients such as solvents or emulsifiers for the formulation of a cream, an ointment, a gel, a paste, or an eye drop.

In some embodiments, provided pharmaceutical compositions are sterilized by conventional sterilization techniques, e.g., may be sterile filtered. Resulting aqueous solutions may be packaged for use as is, or lyophilized. Lyophilized preparations can be, for example, combined with a sterile aqueous carrier prior to administration. The pH of preparations typically will be between 3 and 11, more preferably between 5 and 9 or between 6 and 8, and most preferably between 6 and 7, such as 6 to 6.5. Resulting compositions in solid form may be packaged, for example, in multiple single dose units, each containing a fixed amount of the above-mentioned agent or agents, such as in a sealed package of tablets or capsules. Pharmaceutical compositions in solid form can also be packaged in a container for a flexible quantity, such as in a squeezable tube designed for a topically applicable cream or ointment.

### Functional Effects

In many embodiments, administering radioimmunoconjugates in accordance with provided methods results in infiltration of a lymphocyte population into a tumor (e.g., into the core of a tumor). In some embodiments, the lymphocyte population comprises T cells (e.g., CD8+ T cells). In some embodiments, the lymphocyte population comprises cytotoxic lymphocytes, e.g., activated CD8+ T cells and/or natural killer cells. In some embodiments, the lymphocyte population comprises cells that are positive for one or more markers (cell surface markers and/or intracellular markers) of cytotoxic and/or activated lymphocytes. In some embodiments, the lymphocyte population comprises cells that are positive for markers selected from the group consisting of CD3, CD8, CD16, CD25, CD27, CD44, CD45, CD46, CD53, CD56, CD57, CD69, CD137, Granzyme B, or a combination thereof.

In some embodiments, the lymphocyte population comprises a CD8+ T cell population which comprises CD8+ T cells expressing a T cell receptor specific for (e.g., recognizes) a tumor-associated antigen. In some embodiments, the tumor-associated antigen is different than the tumor-associated antigen to which the targeting moiety within the radioimmunoconjugate is capable of binding. In some embodiments, the CD8+ T cells express a T cell receptor specific for a tumor-associated antigen that is a neoantigen.

In some embodiments, the lymphocyte population comprises cells (e.g., CD8+ T cells) capable of preferentially killing a cell expressing a tumor-associated antigen.

In some embodiments, the lymphocyte population (e.g., CD8+ T cell population) is detectable in the tumor (e.g., in an area of the tumor such as the core of the tumor) at a level greater than a reference level, e.g., at least two-fold, at least three-fold, at least four-fold, or at least five-fold greater than the reference level.

In some embodiments, the lymphocyte population (e.g., CD8+ T cell population) represents at least 5%, at least 7.5%, at least 10%, at least 12.5%, or at least 15% of the cells in the tumor or an area of the tumor (e.g., in the core of the tumor).

In some embodiments, CD8+ T cells expressing a T cell receptor specific for (e.g., recognizes) a tumor-associated antigen represent at least 15%, at least 20%, at least 25%, at least 30%, at least 35%, at least 40%, at least 45%, at least 50%, at least 55%, at least 60%, at least 65%, or at least 70% of the CD8+ T cell population that has infiltrated the tumor or an area of the tumor (e.g., the core of the tumor).

In some embodiments, CD8+ cells (e.g., CD8+ cells expressing a T cell receptor specific for a tumor-associated antigen is detectable in the subject (e.g., detectable in a tissue or sample thereof of the subject) at least 15 days, at least 20 days, at least 25 days, at least 30 days, at least 35 days, or at least 40 days after the step of administering.

In certain embodiments, administering radioimmunoconjugates in accordance with provided methods results in inhibition of cell proliferation in the tumor, e.g., in the core of the tumor. For example, in some embodiments, cell proliferation in tumor tissues from administered subjects is lower by at least 1.5-fold, at least 2-fold, at least 3-fold, at least 4-fold, at least 5-fold, at least 6-fold, at least 7-fold, at least 8-fold, at least 9-fold, at least 10-fold, at least 12.5 fold, at least 15-fold, at least 17.5 fold, or at least 20-fold relative to a reference level. Cell proliferation may be assessed using any of a variety of methods known in the art, including assessment of markers (e.g., Ki67) in or on cells.

Numbers and/or proportions of cells that are positive for a particular marker (e.g., a marker of CD8 T cells, a marker of cell proliferation, a marker of apoptosis, etc.) in a tumor may be assessed using any of a variety of known methods. For example, in some methods, the number of cells that stain positive for the marker in a cross section are calculated as a percentage of all cell nuclei (e.g., nuclei staining positive for a marker of viable cells) in an area within a cross section of the tumor. In certain embodiments, counting is performed in several areas within a cross section (e.g., at least three, at least four, or at least five areas), and a percentage is calculated as an average of the counts from various areas.

In some embodiments, hypoxic areas within tumors (e.g., areas typically found in the center of tumors) are excluded when performing cell counting. These hypoxic areas tend to be necrotic in the absence of any therapeutic agent. For example, when assessing tumor cell death (e.g., by apoptosis and/or necrosis) that occurs as a result of an agent administered to a subject with a tumor, areas other than such hypoxic areas may be chosen to perform cell counting.

In some methods, cells are quantitated by obtaining or preparing single-cell suspensions from a tissue of interest, staining cells for one or more markers, and performing a cell sorting or quantification technique on the stained cells, such as using as flow cytometry.

In certain embodiments, administering radioimmunoconjugates in accordance with provided methods results in a slowing or inhibition of progression of the tumor. In some embodiments, the step of administering results in regression of the tumor. In some embodiments, the step of administering results in complete regression of the tumor.

In certain embodiments, administering radioimmunoconjugates in accordance with provided methods results in inhibition of metastasis of tumor cells. Such inhibition may include, for example, reduction of the number of metastases, reduction in the aggressiveness of metastases, and/or delay in the development of metastases.

### Tumors

The patient in need of treatment may have one or more tumors. The one or more tumors may include a primary tumor, a secondary tumor, or both a primary tumor and a secondary tumor.

In some embodiments, the tumor is not highly immunogenic, e.g., the tumor is moderately immunogenic or immunologically cold. In some embodiments, the tumor is not responsive or only partially responsive to checkpoint inhibitor therapy. In some embodiments, the tumor (in the absence of treatment) is characterized by no or low infiltration of lymphocytes (e.g., T cells such as CD8+ T cells), e.g., at levels less than 10%, less than 7.5%, less than 5%, less than 4%, less than 3%, less than 2%, less than 1%, or less than 0.5% of total viable cells or cell nuclei.

In some embodiments, the tumor is at least 50 mm³, at least 75 mm³, at least 100 mm³, at least at least 125 mm³, at least 150 mm³, at least 175 mm³, or at least 200 mm³ in volume at the time of administering the radioimmunoconjugate.

In some embodiments, the cancer is a solid tumor, e.g., a carcinoma, sarcoma, melanoma, or lymphoma.

In some embodiments, the solid tumor is a carcinoma, e.g., adenocarcinoma, squamous cell carcinoma, or adenosquamous carcinoma. Non-limiting examples of carcinomas include adenoid cystic carcinoma, adrenocortical carcinoma, bladder cancer, breast cancer, cervical cancer, colorectal cancer, endometrial cancer, gallbladder carcinoma, gastric cancer, head and neck cancer, lung cancer (e.g., small cell lung cancer or non-small cell lung cancer, or adenocarcinoma of the lung), neuroblastoma, neuroendocrine cancer, ovarian cancer, pancreatic cancer, prostate cancer, renal cancer, testicular cancer.

In some embodiments, the solid tumor is a sarcoma. Non-limiting examples of sarcomas include angiosarcoma or hemangioendothelioma, astrocytoma, chondrosarcoma, Ewing's sarcoma, fibrosarcoma, glioma, leiomyosarcoma, liposarcoma, malignant fibrous histiocytoma (MFH), mesenchymous or mixed mesodermal tumor, mesothelial sarcoma or mesothelioma, myxosarcoma, osteosarcoma, rhabdomyosarcoma, and synovial sarcoma.

In some embodiments, the tumor is a neuroblastoma.

In some embodiments, the tumor is a brain tumor. In some embodiments, the tumor is a glioma.

In some embodiments, the tumor is a melanoma.

In some embodiments, the tumor is a non-solid tumor, e.g., a liquid tumor or hematologic tumor. In some embodiments, the tumor is a myeloma, e.g., multiple myeloma. In some embodiments, the tumor is a leukemia, e.g., acute myeloid leukemia.

In some embodiments, the tumor is a mixed type tumor, e.g., mixed mesodermal tumor, carcinosarcoma, or teratocarcinoma.

### Subjects

In some embodiments, the subject is a mammal, e.g., a human.

In some embodiments, the subject has cancer or is at risk of developing cancer. For example, the subject may have been diagnosed with cancer. For example, the cancer may be a primary cancer or a secondary (e.g., metastatic) cancer. Subjects may have any stage of cancer, e.g., stage I, stage II, stage III, or stage IV with or without lymph node involvement and with or without metastases. Provided radioimmunoconjugates and compositions may prevent or reduce further growth of the cancer and/or otherwise ameliorate the cancer (e.g., prevent or reduce metastases). In some embodiments, the subject does not have cancer but has been determined to be at risk of developing cancer, e.g., because of the presence of one or more risk factors such as environmental exposure, presence of one or more genetic mutations or variants, family history, etc. In some embodiments, the subject has not been diagnosed with cancer.

In some embodiments, the subject is in need of treatment of a refractory cancer.

### Administration and Dosage

Radioimmunoconjugates described herein and pharmaceutical compositions thereof may be administered by any of a variety of routes of administration, including systemic and local routes of administration

Systemic routes of administration include parenteral routes and enteral routes. In some embodiments, radioimmunoconjugates or pharmaceutical compositions thereof are administered by a parenteral route, for example, intravenously, intraarterially, intraperitoneally, subcutaneously, or intradermally. In some embodiments, radioimmunoconjugates or pharmaceutical compositions thereof are administered intravenously. In some embodiments, radioimmunoconjugates or pharmaceutical compositions thereof are administered by an enteral route of administration, for example, trans-gastrointestinal, or orally.

Local routes of administration include, but are not limited to, peritumoral injections and intratumoral injections.

Pharmaceutical compositions can be administered for radiation treatment planning, diagnostic, and/or therapeutic treatments. When administered for radiation treatment planning or diagnostic purposes, the radioimmunoconjugate may be administered to a subject in a diagnostically effective dose and/or an amount effective to determine the therapeutically effective dose. In therapeutic applications, pharmaceutical compositions may be administered to a subject (e.g., a human) already suffering from a condition (e.g., cancer) in an amount sufficient to cure or at least partially arrest the symptoms of the disorder and its complications. An amount adequate to accomplish this purpose is defined as a “therapeutically effective amount,” an amount of a compound sufficient to substantially improve at least one symptom associated with the disease or a medical condition. For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but may, for example, provide a treatment for a disease or condition such that the onset of the disease or condition is delayed, hindered, or prevented, such that the disease or condition symptoms are ameliorated, or such that the term of the disease or condition is changed. For example, the disease or condition may become less severe and/or recovery is accelerated in an individual. In some embodiments, a subject is administered a first dose of a radioimmunoconjugate or composition in an amount effective for radiation treatment planning, then administered a second dose or set of doses of the radioimmunoconjugate or composition in a therapeutically effective amount.

Effective amounts may depend on the severity of the disease or condition and other characteristics of the subject (e.g., weight). Therapeutically effective amounts of disclosed radioimmunoconjugates and compositions for subjects (e.g., mammals such as humans) can be determined by the ordinarily-skilled artisan with consideration of individual differences (e.g., differences in age, weight, and the condition of the subject.

In some embodiments, radioimmunoconjugates exhibit an enhanced ability to target cancer cells. In some embodiments, effective amount of disclosed radioimmunoconjugates are lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose for a therapeutic effect of the unconjugated, and/or non-radiolabeled targeting moiety.

Single or multiple administrations of pharmaceutical compositions herein including an effective amount can be carried out with dose levels and pattern being selected by the treating physician. Dose and administration schedule can be determined and adjusted based on the severity of the disease or condition in the subject, which may be monitored throughout the course of treatment according to the methods commonly practiced by clinicians or those described herein.

### Therapeutic Regimens

In certain embodiments, the radioimmunoconjugate is the only cytotoxic agent administered as part of a therapeutic regimen. For example, in some embodiments, the radioimmunoconjugate is not administered in combination with (whether simultaneously, sequentially, or in overlapping dosing regions) another cytotoxic agent. Thus, in these embodiments, the only cytotoxic agent to which the subject is exposed during the course of a therapeutic regimen comprising the radioimmunoconjugate is the radioimmunoconjugate itself.

In some embodiments, the radioimmunoconjugate is administered in combination with another agent, e.g., a therapeutic agent. In some embodiments, the other therapeutic agent is a non-cytotoxic agent, e.g., a DNA damage and repair inhibitor (DDRi), a checkpoint inhibitor, or any combination thereof.

### Checkpoint Inhibitors

In some embodiments, a checkpoint inhibitor is administered in combination with a radioimmunoconjugate. Generally, suitable checkpoint inhibitors inhibit an immune suppressive checkpoint protein. In some embodiments, the checkpoint inhibitor inhibits a protein selected from the group consisting of cytotoxic T-lymphocyte-associated antigen 4 (CTLA-4), programmed death 1 (PD-1), programmed death ligand-1 (PD-L1), LAG-3, T cell immunoglobulin mucin 3 (TIM-3), and killer immunoglobulin-like receptors (KIRs).

For example, in some embodiments, the checkpoint inhibitor is capable of binding to CTLA-4, PD-1, or PD-L1. In some embodiments, the checkpoint inhibitor interferes with the interaction (e.g., interferes with binding) between PD-1 and PD-L1.

In some embodiments, the checkpoint inhibitor is a small molecule.

In some embodiments, the checkpoint inhibitor is an antibody or antigen-binding fragment thereof, e.g., a monoclonal antibody. In some embodiments, the checkpoint inhibitor is a human or humanized antibody or antigen-binding fragment thereof. In some embodiments, the checkpoint inhibitor is a mouse antibody or antigen-binding fragment thereof.

In some embodiments, the checkpoint inhibitor is a CTLA-4 antibody. Non-limiting examples of CTLA-4 antibodies include BMS-986218, BMS-986249, ipilimumab, tremelimumab (formerly ticilimumab, CP-675,206), MK-1308, and REGN-4659. An additional example of a CTLA-4 antibody is 4F10-11, a mouse monoclonal antibody.

In some embodiments, the checkpoint inhibitor is a PD-1 antibody. Non-limiting examples of PD-1 antibodies include camrelizumab, cemiplumab, nivolumab, pembrolizumab, sintilimab, tislelizumab and toripalimab. An additional example of a PD-1 antibody is RMP1-14, a mouse monoclonal antibody.

In some embodiments, the checkpoint inhibitor is a PD-L1 antibody. Non-limiting examples of PD-L1 antibodies include atezolizumab, avelumab, and durvalumab.

In some embodiments, a combination of more than one checkpoint inhibitor is used. For example, in some embodiments, both a CTLA-4 inhibitor and a PD-1 or PD-L1 inhibitor is used.

Without further elaboration, it is believed that one skilled in the art can, based on the above description, utilize the present disclosure to its fullest extent. The following specific examples are therefore to be construed as merely illustrative, and not limitative of the remainder of the disclosure in any way whatsoever.

### EXAMPLES

### Example 1. Synthesis of [²²⁵Ac]-FPI-1792, an IGF-1R-Targeting Radioimmunoconjugate

TAB-199 (a human monoclonal IGF-1R antibody that also recognizes murine IGF-1R with subnanomolar affinity, available commercially from Creative Biolabs,) was conjugated to FPI-1397 (Fusion Pharmaceuticals), a bifunctional chelate comprising a 1,4,7,10-tetraazacyclododecane-1,4,7,10-tetraacetic acid (DOTA) chelating moiety and a FastClear™ linker (Fusion Pharmaceuticals). The structure of FPI-1397 is shown below:

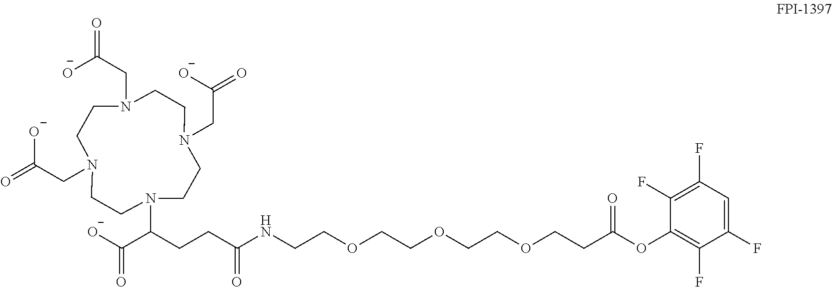 [SMILES: O=C([O-])CN1CCN(CC(=O)[O-])CCN(C(CCC(=O)NCCOCCOCCOCCC(=O)Oc2c(F)c(F)cc(F)c2F)C(=O)[O-])CCN(CC(=O)[O-])CC1]

The resulting conjugate was purified, reformulated, and radiolabeled with [²²⁵Ac]. The final conjugate, named [²²⁵Ac]-FPI-1792, comprises the following structure, with TAB-199 as the antibody on the right-hand side. [²²⁵Ac] is complexed to the DOTA moiety.

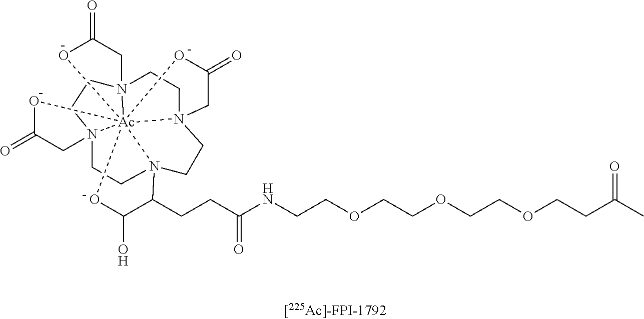

### Example 2. Core Tumor Infiltration by CD8+ T Cells after Administration of [²²⁵Ac]-FPI-1792 in a Moderately Immunogenic Colon Cancer Cell Line

[²²⁵Ac]-FPI-1792 is a radioimmunoconjugate comprising TAB-199 conjugated to a 1,4,7,10-tetraazacyclododecane-1,4,7,10-tetraacetic acid (DOTA) chelating moiety via a Fast-Clear™ linker, with the ²²⁵Ac complexed to the DOTA moiety.

Effects of administering [²²⁵Ac]-FPI-1792 on tumor cell proliferation, and tumor growth were evaluated in a moderately immunogenic syngeneic mouse model, a CT-26 mouse model of colon cancer. CT-26 cells express murine IGF-1R and are sensitive to anti-CTLA-4 antibodies but only partially sensitive to anti-PD-1 antibodies.

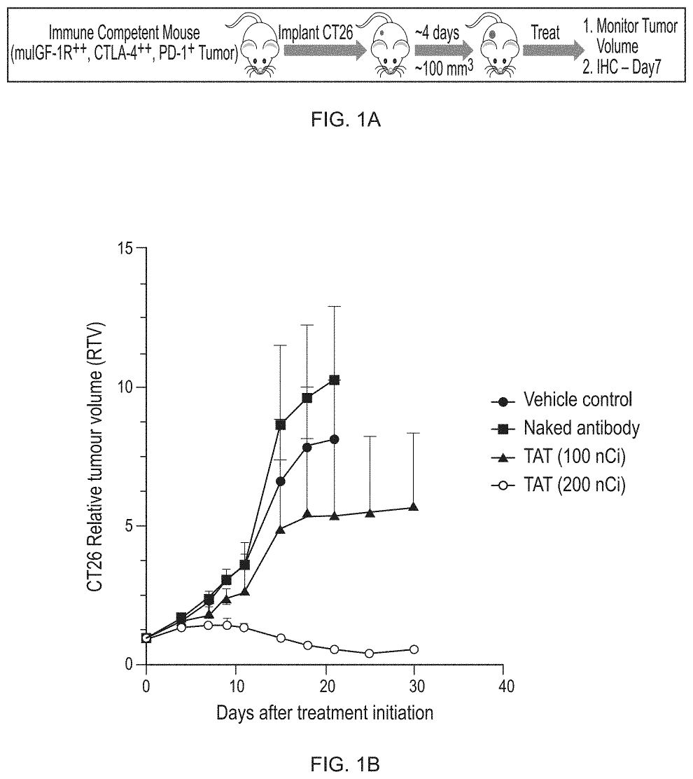

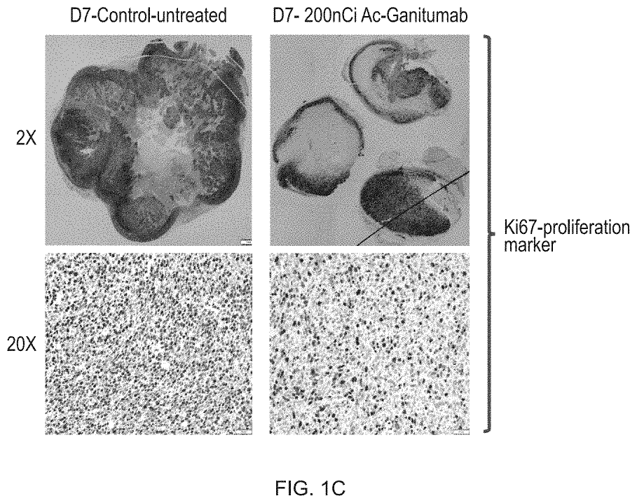

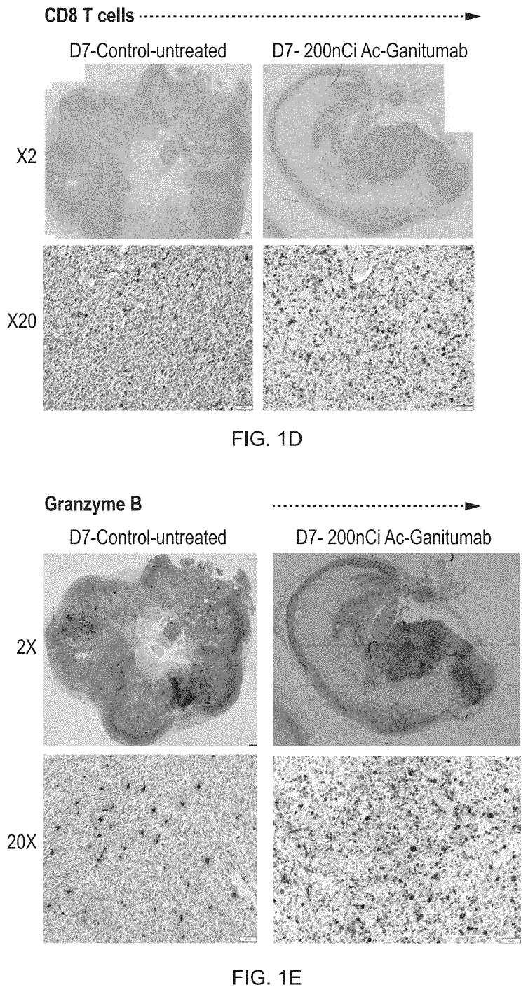

FIG. 1A depicts a schematic of these experiments. CT-26 cells were implanted into immune competent mice. Implanted CT-26 cells were allowed to proliferate in situ for 4 days or until they produced tumors of a volume of approximately 100-150 mm³. Animals were then administered vehicle (control), TAB-199 (naked antibody), or [²²⁵Ac]-FPI-1792 (100 nCi or 200 nCi).

CT-26 tumor volume was monitored after treatment initiation. Relative tumor volume of CT-26 tumors in animals administered vehicle control or naked antibody increased between 0 and 20 days after administration (FIG. 1B). Administration of 100 nCi of TAT resulted in a relatively slower increase in tumor growth and small relative tumor size between days 7 and 20 compared to administration of vehicle control or naked antibody (FIG. 1B). Administration of 100 nCi of [²²⁵Ac]-FPI-1792 resulted in a plateau in relative tumor volume on or about day 18 post-administration (FIG. 1B). Administration of 200 nCi of [²²⁵Ac]-FPI-1792 resulted in smaller relative tumor size between days 7 and 20 compared to administration of vehicle control, naked antibody, or 100 nCi [²²⁵Ac]-FPI-1792 (FIG. 1B). Additionally, administration of 200 nCi [²²⁵Ac]-FPI-1792 resulted in a relative decrease in tumor volume compared to baseline (FIG. 1B). Specifically, after approximately 9 days post-administration, CT-26 tumors of animals treated with TAT at 200 nCi began to decrease in size relative to the size of CT-26 tumors at the start of treatment. The decrease in CT-26 tumor volume in animals treated with TAT at 200 nCi appeared to continue until about 25 days post-administration, after which tumor volume remained relatively unchanged (FIG. 1B).

CT-26 tumors were harvested from animals 7 days post-administration to evaluate tumor cell proliferation and infiltration of CD8⁺ T cells into tumor cores by immunohistochemistry. Briefly, Formalin-Fixed Paraffin-Embedded (FFPE) tissue was prepared for immunohistochemistry as follows: tumors were excised, fixed in formaldehyde, embedded paraffin, and sectioned.

In cross sections, areas from the tumor core (>250 μM from the margin boundary) and away from hypoxic/necrotic areas were evaluated.

Tumor cell proliferation was evaluated by immunostaining for the cell proliferation marker Ki67. Ki67 staining of tumor tissue from CT-26-injected animals administered 200 nCi [²²⁵Ac]-FPI-1792 showed lower levels of Ki67 staining as compared to tumor tissue from CT-26-injected animals administered vehicle (FIG. 1C), suggesting that administration of [²²⁵Ac]-FPI-1792 reduced tumor cell proliferation.

Infiltration of CD8⁺ T cells into CT-26 tumor cores was evaluated by immunostaining for CD8. CD8 staining of tumor tissue from CT-26-injected animals administered 200 nCi [²²⁵Ac]-FPI-1792 showed relatively higher levels of CD8 staining as compared to tumor tissue from CT-26-injected animals administered vehicle (FIG. 1D). Functionality of cytotoxic T cells and natural killer cells in CT-26 tumors was evaluated by immunostaining for Granzyme B, a serine protease expressed in cytotoxic T cells and natural killer cells. Granzyme B staining of tumor tissue from CT-26-injected animals treated 200 nCi [²²⁵Ac]-FPI-1792 showed relatively higher levels of Granzyme B staining as compared to tumor tissue from CT-26-injected animals administered vehicle (FIG. 1E). These results suggest significant tumor core infiltration of CD8+ T cells, including activated CD8+ T cells and/or natural killer cells.

These results demonstrate that administering [²²⁵Ac]-FPI-1792, an alpha-emitting radioimmunoconjugate capable of recognizing IGF-1R, resulted in infiltration of CD8⁺ T cells and other Granzyme B-expressing immune cells into tumor cores at markedly increased levels relative to levels obtained when administering a vehicle control. Administration of the [²²⁵Ac]-FPI-1792 also decreased tumor cell proliferation of IGF-1R-expressing tumor cells, decreased relative tumor growth (compared to administration of vehicle control or cold antibody), and reversed overall tumor growth.

### Example 3. Quantitation of CD8+ T Cells in Core Tumors after Administration of [²²⁵Ac]-FPI-1792 in a Moderately Immunogenic Colon Cancer Cell Line

To assess cell proliferation and CD8+ T cell tumor core infiltration further, CT-26 tumor inoculation and treatment according to one of five treatment groups (shown below) was performed as described in Example 2, except that CT-26 tumors were allowed to develop to a later stage than in Example 2. Tumors were allowed to develop for approximately 6 days (or until tumors were approximately 175 mm³) before initiation of treatment.
Vehicle
TAB-199 (cold antibody)
200 nCi [²²⁵Ac]-FPI-1792
200 nCi [²²⁵Ac]-FPI-1792+anti-PD1
200 nCi [²²⁵Ac]-FPI-1792+anti-CTLA4
200 nCi [²²⁵Ac]-FPI-1792+anti-PD1+anti-CTLA4

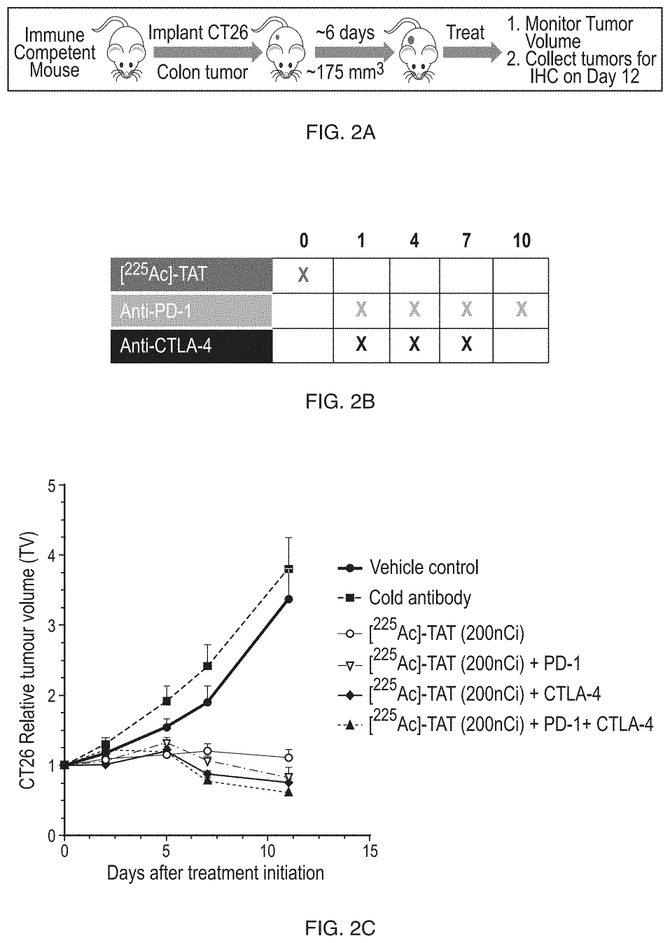

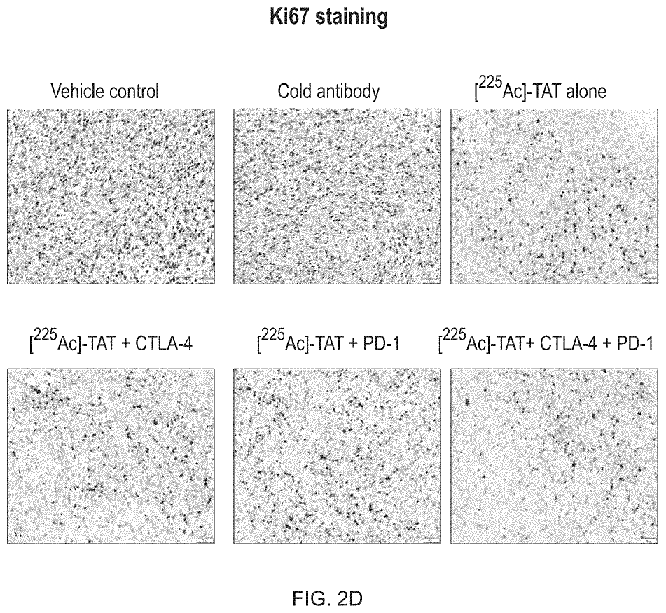

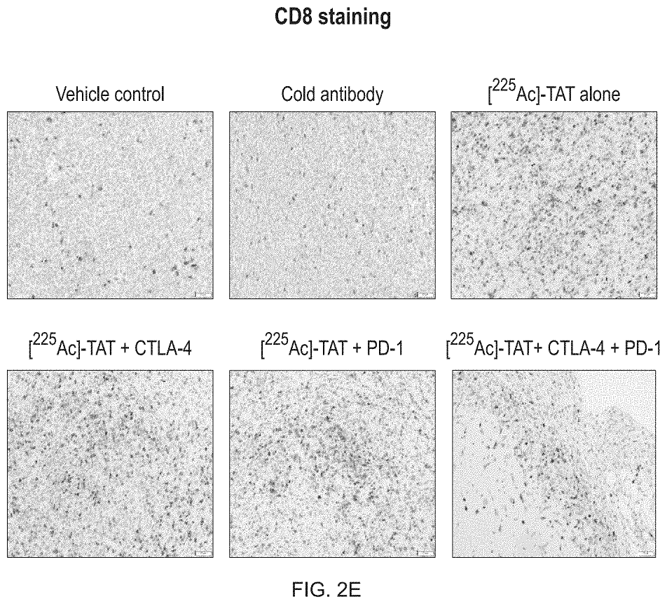

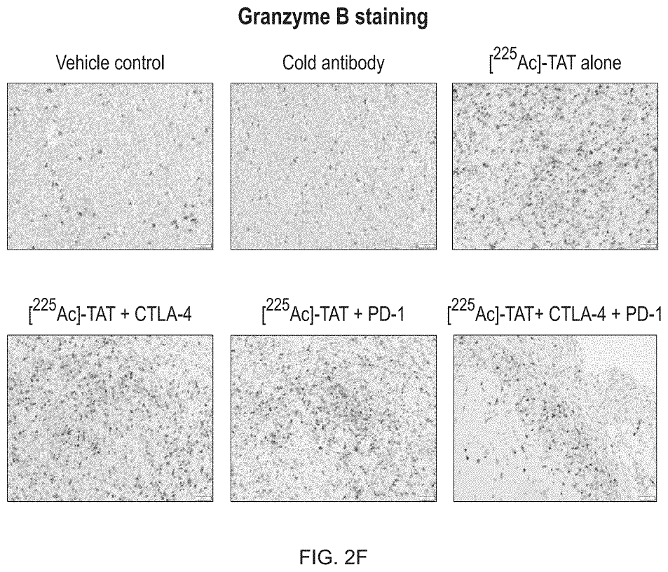

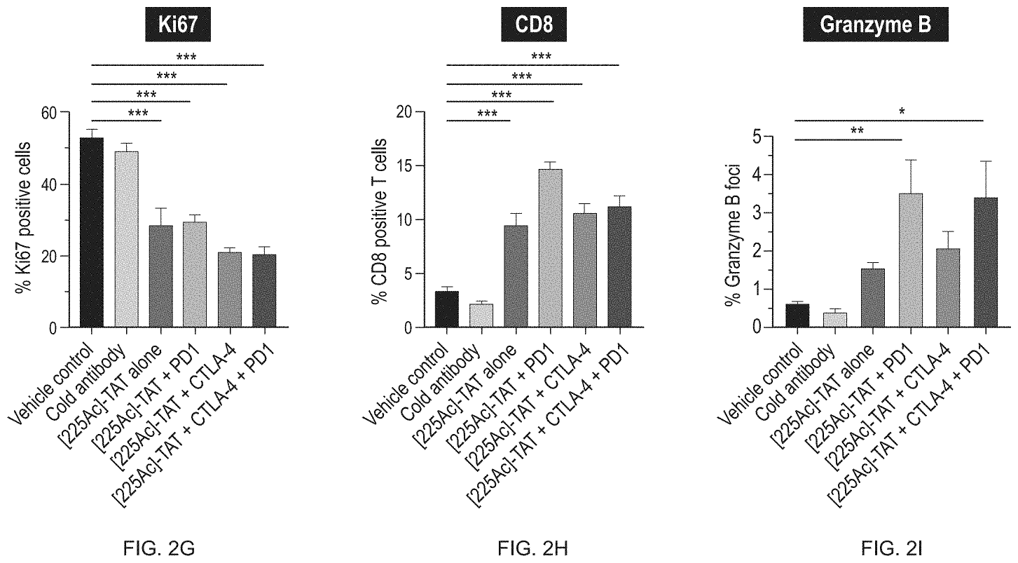

FIG. 2A depicts a schematic of these experiments. FIG. 2B illustrates the dosing schedule (in days) for the various therapeutic agents (if administered). In combination treatment groups, 200 nCi [²²⁵Ac]-FPI-1792 was administered first, with any additional therapeutic agents administered beginning the day after.

As shown in FIG. 2C (depicting relative tumor volume), all treatment groups that included 200 nCi [²²⁵Ac]-FPI-1792 (including the group in which 200 nCi [²²⁵Ac]-FPI-1792 was administered without a co-therapeutic) exhibited significantly reduced tumor growth compared to the tumor growth in the vehicle and cold antibody groups.

Tumors were collected for immunohistochemical analyses on day 12 after treatment initiation, and FFPE tissue was prepared as described in Example 2. Tumor sections were stained for various markers of cell proliferation (Ki67), T-cells (CD8), and cytotoxic T cells or natural killer cells (Granzyme B). Representative non-areas necrotic areas from tumor cores (e.g., >250 um from the margin boundary) are shown in FIGS. 2D, 2E, and 2F.

FIG. 2D shows representative results from sections stained with Ki67. Markedly reduced Ki67 (and therefore reduced cell proliferation) was observed in sections from all treatment groups that included 200 nCi [²²⁵Ac]-FPI-1792, relative to vehicle and cold antibody controls.

FIG. 2E shows representative results from sections stained with CD8. Markedly increased CD8 staining was observed in sections from all treatment groups that included 200 nCi [²²⁵Ac]-FPI-1792, relative to vehicle and cold antibody controls. FIG. 2F shows representative results from sections stained with Granzyme B. Markedly increased Granzyme B staining was observed in sections from all treatment groups that included 200 nCi [²²⁵Ac]-FPI-1792, relative to vehicle and cold antibody controls. These results suggest significant tumor core infiltration of CD8+ T cells, including activated CD8+ T cells and/or natural killer cells.

To quantitate the immunohistochemistry results, numbers of cells that were Ki67-, CD8-, and Granzyme B-positive cells were counted from five separate areas and averaged for each treatment group. Percentages of positive cells were calculated as percentages of all viable cell nuclei.

FIG. 2G depicts quantitation results to determine % Ki67-positive cells. All treatment groups that included 200 nCi [²²⁵Ac]-FPI-1792 showed significantly decreased Ki67 staining (and therefore decreased cell proliferation) compared to vehicle and TAB-199 (cold antibody) controls. No significant differences were detected between the treatment groups that included 200 nCi [²²⁵Ac]-FPI-1792.

FIG. 2H depicts quantitation results to determine % CD8-positive cells. All treatment groups that included 200 nCi [²²⁵Ac]-FPI-1792 showed significantly increased percentages of CD8+ cells compared to vehicle and TAB-199 (cold antibody) controls. No significant differences were detected between the treatment groups that included 200 nCi [²²⁵Ac]-FPI-1792.

FIG. 2I depicts quantitation results to determine % Granzyme B-positive cells. All treatment groups that included 200 nCi [²²⁵Ac]-FPI-1792 showed significantly increased percentages of Granzyme B+ cells compared to vehicle and TAB-199 (cold antibody) controls. No significant differences were detected between the treatment groups that included 200 nCi [²²⁵Ac]-FPI-1792.

These results demonstrate that administering [²²⁵Ac]-FPI-1792 at a later time point (and when tumors were larger) relative to the time of administration for the experiments described in Example 2 also resulted in infiltration of CD8⁺ T cells and other Granzyme B-expressing immune cells into tumor cores, decreased tumor cell proliferation, decreased relative tumor growth (compared to administration of vehicle control or cold antibody), and reversed overall tumor growth.

Additionally, cell quantitation analyses revealed that the differences in infiltration (as assessed by presence of CD8+ and Granzyme B+ staining in the core of the tumor) and cell proliferation were statistically significant between control (vehicle and cold antibody) and treatment ([²²⁵Ac]-FPI-1792 alone or in combination with one or more checkpoint inhibitors) at 12 days after treatment Moreover, at the time point assessed, no treatment with [²²⁵Ac]-FPI-1792 alone yielded comparable CD8+ T cell infiltration and cell proliferation results as did treatment with [²²⁵Ac]-FPI-1792 in combination with one or more checkpoint inhibitors.

### Example 4. Sustained Effects of [²²⁵Ac]-FPI-1792 Administration as Demonstrated in a Tumor Re-Challenge Experiment in a Colorectal Cancer Cell Model

Persistence of tumor-infiltrating lymphocytes was evaluated by assessing tumor growth and presence of CD8+ T cells in secondary tumor cores in mice that had been inoculated with CT-26 colon cancer cells, treated with [²²⁵Ac]-FPI-1792, and subsequently re-challenged with the same line (CT-26) of tumor cells.

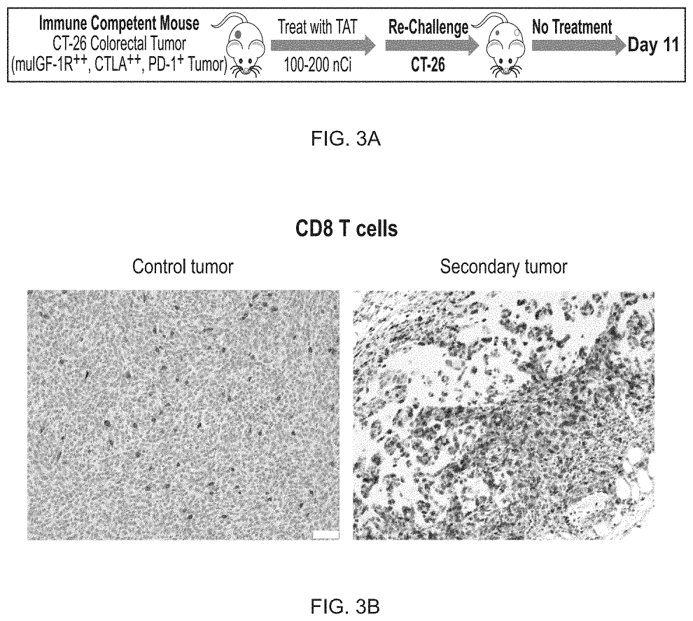

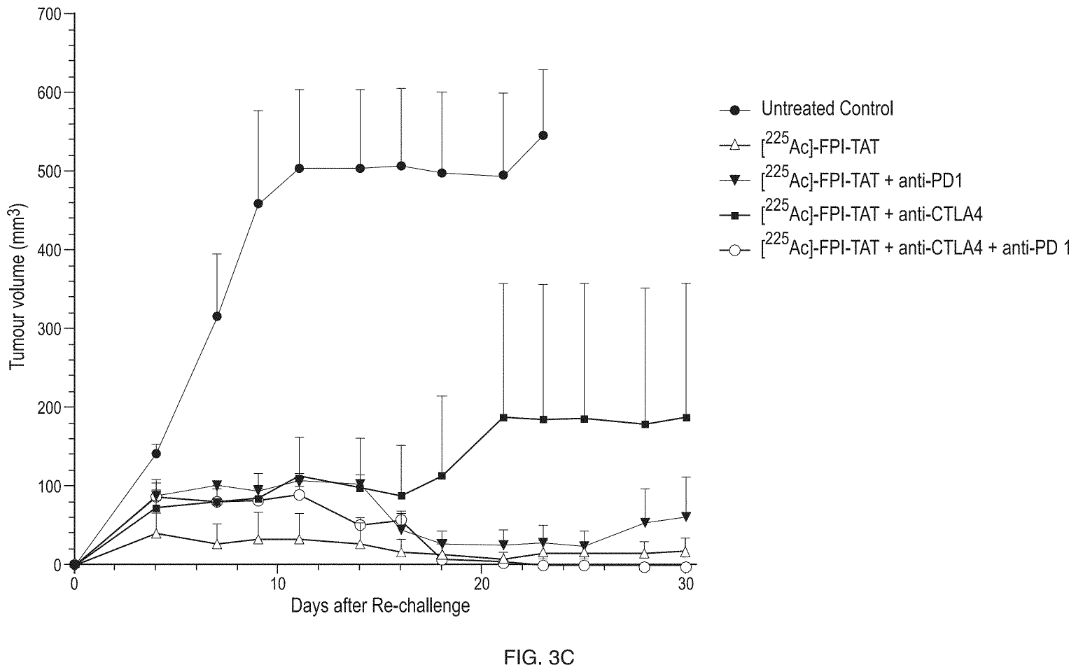

FIG. 3A outlines the schematic for this re-challenge experiment.

CT-26 tumor inoculation and treatment with [²²⁵Ac]-FPI-1792 was performed as described in Example 2. Additional groups of mice were treated with: 1) [²²⁵Ac]-FPI-1792+anti-PD1; 2) [²²⁵Ac]-FPI-1792+anti-CTLA4; or 3)) [²²⁵Ac]-FPI-1792+anti-PD1+anti-CTLA4. Thirty days after administration of treatment, animals were re-challenged with allograft implantation of CT-26 cells on the contralateral flank. No treatment was given after re-challenge. Untreated mice were used as controls.

Eleven days after re-challenge, tissues were obtained from secondary tumors. FFPE tissue was prepared as described in Example 2. Areas from tumor cores were evaluated for reactivity with a CD8 antibody. Relative to controls, higher frequencies of CD8+ T cells were detected in secondary tumor tissue from animals originally treated with 200 nCi [²²⁵Ac]-FPI-1792 alone and subsequently re-challenged with CT-26 cells (FIG. 3B). These results suggest that a single administration of [²²⁵Ac]-FPI-1792 induces a CD8+ T cell population that is capable of infiltrating the cores of secondary tumors. Moreover, this CD8+ T cell population persists at levels higher relative to a control.

Tumor growth after re-challenge was assessed in all treatment groups and controls. As shown in FIG. 3C, all treatment groups showed markedly reduced tumor growth relative to controls. Thirteen of 15 animals in various treatment groups (treated with [²²⁵Ac]-FPI-1792 alone or in combination with one or more checkpoint inhibitors) showed no growth of secondary tumor.

These results demonstrate a “vaccine effect” mediated by a single administration of [²²⁵Ac]-FPI-1792 alone, or in combination with checkpoint inhibitors. Thus, [²²⁵Ac]-FPI-1792 administration alone confers a sustained and beneficial effect via CD8+ T cells that persist and ultimately infiltrate into cores of secondary tumor.

These results suggest that treatment with [²²⁵Ac]-FPI-1792 may facilitate amelioration or prevention of tumor recurrences or secondary metastases.

### Example 5. [²²⁵Ac]-FPI-1792-Mediated Recruitment of Tumor Antigen-Specific CD8+ T Cells from Spleen to Tumors

Recruitment of T-cells from the spleen to secondary tumors was assessed in mice presented with a CT-26 tumor re-challenge in the experiment described in Example 4.

As mentioned in Example 4, no treatment was given to mice after tumor re-challenge. At day 14 after re-challenge, secondary tumors and spleens were isolated from mice in the following treatment groups:
untreated (vehicle)
[²²⁵Ac]-FPI-1792+anti-PD1
[²²⁵Ac]-FPI-1792+anti-CTLA4
[²²⁵Ac]-FPI-1792+anti-PD1+anti-CTLA4

Tissues were digested with collagenase and DNase to form a single cell suspension. CD45+ hematopoietic cells were purified from the single cell suspension by magnetic selection, then cells were stained and analyzed for expression of CD8.

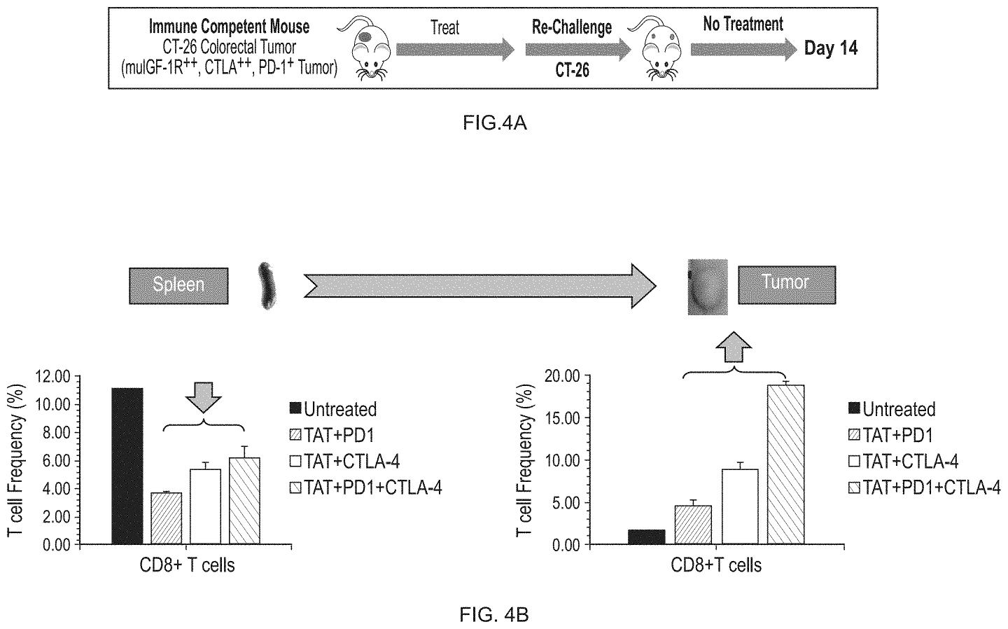

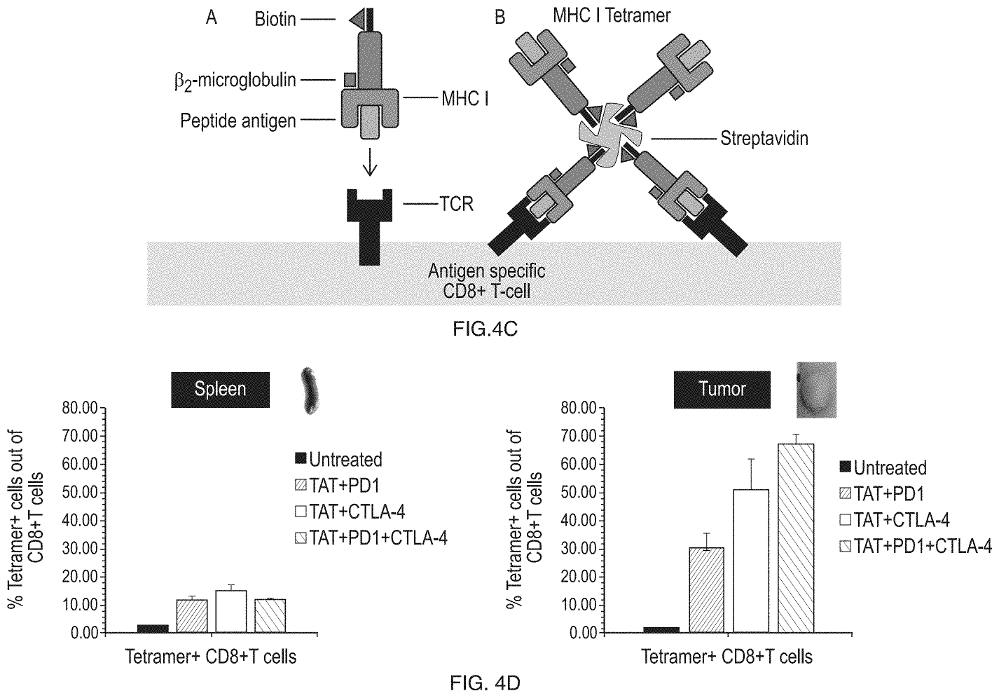

FIG. 4B shows T cell frequencies (as assessed by CD8 expression in CD45+ purified cells) in spleen (left panel) and tumor (right panel) cells across analyzed treatment groups. Relative to corresponding samples from vehicle controls, spleen samples in the [²²⁵Ac]-FPI-1792 combination therapy treatment groups exhibited reduced levels of CD8+ T Cells, whereas tumor samples in the [²²⁵Ac]-FPI-1792 combination therapy treatment groups exhibited increased levels of CD8+ T cells. These results indicate extensive recruitment of CD8+ T cells into secondary tumors after re-challenge.

To assess the proportion of CD8+ T cells that were specific for tumor cells, an MHC I tetramer-based analysis was performed using AH1 (SPSYVYHGF (SEQ ID NO: 1)), an immunodominant CD8+ T cell epitope from CT26. FIG. 4C is a schematic showing this tetramer technique. Tetramer reagent is assembled from four identical biotinylated MHC class I/β2M units, loaded with AH1 peptide antigen and held together with fluorescently-labelled streptavidin to allow detection of antigen-specific T cells by flow cytometry. Thus, tetramer-positive T cells are T cells expressing a T-cell receptor specific for a CT26 epitope.

FIG. 4D presents tetramer analysis results from spleen (left panel) and tumor (right panel) cells across analyzed treatment groups. Levels of tetramer cells are presented as percentages of CD8+ T cells. In the spleen, increased levels of CT26 epitope-specific T cells were detected in treated mice (approximately 11-17%) as compared to untreated controls (2-3%). In the tumor, very high frequencies of CT26 epitope-specific T cells were detected in the tumors of treated mice (approximately 30-70%) as compared to untreated controls (1-2%).

These results indicate extensive accumulation of tumor-specific CD8+ T cells in secondary tumors. Moreover, these results indicate that administration of [²²⁵Ac]-FPI-1792 leads to production and infiltration of CD8+ T cells with T-cell receptors specific for a tumor-associated antigen expressed by tumor cells.

### Example 6. Tumor Suppression in an Immunologically Cold Metastatic Breast Cancer Cell Line

[²²⁵Ac]-FPI-1792 was assessed in an immunologically cold tumor using the syngeneic 4T1 triple-negative breast cancer model. 4T1 cells express murine IGF-1R and develop tumors with a rapid progression. 4T1 cells are highly metastatic, poorly immunogenic, and resistant to checkpoint inhibition (e.g., with PD1 or CTLA4 blockade).

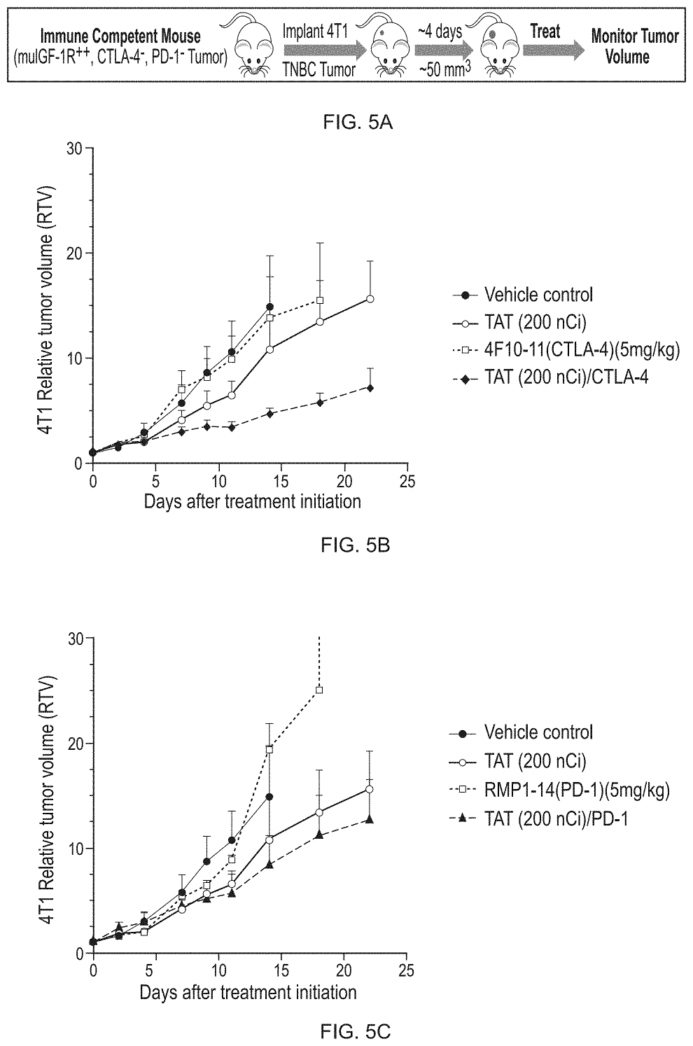

FIG. 5A depicts a schematic of these experiments. 4T1 cells were implanted into Balb/c immune competent mice. Implanted 4T1 cells were allowed to proliferate in situ for 4 days or until they produced tumors of a volume of approximately 50 mm³. Animals were then administered vehicle (control), 200 nCi [²²⁵Ac]-FPI-1792 alone, 200 nCi [²²⁵Ac]-FPI-1792+5 mg/kg 4F10-11 (anti-CTLA4), or 200 nCi [²²⁵Ac]-FPI-1792+5 mg/kg RMP1-14 (anti-PD1).

4T1 tumor volume was evaluated after treatment initiation. FIGS. 5B and 5C show relative tumor volumes for 200 nCi [²²⁵Ac]-FPI-1792 compared against its combination with anti-CTLA4 (FIG. 5B) or against its combination with anti-PD1 (FIG. 5C). Whereas anti-CTLA4 treatment alone did not have an observable effect on tumor growth, significant tumor suppression was observed in the [²²⁵Ac]-FPI-1792+anti-CTLA4 treatment group (FIG. 5B).

These results suggest that [²²⁵Ac]-FPI-1792 can render an immunologically cold tumor susceptible to immune checkpoint inhibition.

### Example 7. A Phase I Clinical Trial to Evaluate Efficacy and Safety of Radioimmunoconjugate Administration

A Phase I clinical trial was conducted to evaluate the safety and tolerability of radioimmunoconjugates for administration to human patients. [²²⁵Ac]-FPI-1434 is a radioimmunoconjugate that includes a humanized monoclonal antibody that binds to IGF-1R (AVE1642) linked via the FastClear™ linker to a DOTA moiety; thus, [²²⁵Ac]-FPI-1434 is similar to [²²⁵Ac]-FPI-1792 in the linker and chelating moiety but differs in the IGF-1R antibody used.

[¹¹¹In]-FPI-1547 is an indium-111 analog of [²²⁵Ac]-FPI-1434 that includes the same linker and antibody as [²²⁵Ac]-FPI-1434. [¹¹¹In]-FPI-1547 is used for patient selection and quantification of IGF-1R expressing targets prior to treatment with [²²⁵Ac]-FPI-1434.

185 MBq of [¹¹¹In]-FPI-1547 was intravenously administered to patients, after which the absorbed radiation dose was estimated. Serial anterior/posterior scintigraphic images of patients were obtained over 6-8 days. Count data was extracted from the whole body scans and CT-based volumes were used to estimate radiation absorbed dose to normal organs and tissues per the MIRD schema. Radiation dose estimates for planned therapeutic administrations of [²²⁵Ac]-FPI-1434 to each patient were then performed using OLINDA/EXM software (version 2.0) and verified to be within protocol-specified radiation dose limits for the kidneys (18 Gy), liver (31 Gy) and lungs (16.5 Gy). Planned therapeutic administrations of [²²⁵Ac]-FPI-1434 followed a modified 3+3 dose escalation design of 5 initial cohorts of 10, 20, 40, 80 and 120 kBq/kg body-weight up to a maximum tolerated dose.

8 patients were administered 185 MBq of [¹¹¹In]-FPI-1547 each and underwent subsequent dosimetric evaluation. All 8 patients demonstrated tumor avidity and all were eligible to receive up to 120 kBq/kg based on dosimetry. Estimated mean radiation doses (±SD) per unit of administered activity for the following organs were: kidneys, 966±179 mGy-Eq/MBq; liver, 803±260 mGy-Eq/MBq; and lungs, 672±185 mGy-Eq/MBq. Mean total body radiation dose (±SD) for all patients was 146±19 mGy-Eq/MBq (range 117-170 mGy-Eq/MBq). Seven (88%) patients received one therapeutic administration ranging from 0.80 to 2.3 MBq [²²⁵Ac]-FPI-1434. [²²⁵Ac]-FPI-1434 was generally well tolerated with no unexpected clinically-significant adverse events reported.

These results demonstrate that administration of a patient-specific dose of an actinium-225 radioimmunoconjugate to human patients was also well tolerated and did not result in unexpected clinically-significant adverse events. Additionally, administration of an indium-Ill radioimmunoconjugate was well-tolerated by human patients and resulted in acceptable levels of radiation in patient bodies overall and in critical organs such as kidneys, liver and lungs.

Thus, both indium-111 and actinium-225 radioimmunoconjugates were safely administered to patients without the occurrence of unexpected clinically-significant adverse events. These results further demonstrate that indium-111 radioimmunoconjugate administration was successfully used to estimate potential risk to patients and to produce patient-specific treatment plans for administration of actinium-225 radioimmunoconjugates.

### EQUIVALENTS/OTHER EMBODIMENTS

Those skilled in the art will recognize or be able to ascertain using no more than routine experimentation, many equivalents to the specific embodiments described herein. Such equivalents are intended to be encompassed by the following claims.

1. A method of inducing CD8+ T cell infiltration into a tumor in a subject in need thereof, wherein the method comprises a step of administering to the subject a radioimmunoconjugate or a pharmaceutical composition thereof, wherein the radioimmunoconjugate comprises the following structure:
A-L-B Formula I-a
wherein
A is a metal complex of a chelating moiety, wherein the metal complex comprises Actinium-225 (²²⁵Ac) or a progeny thereof,
L is a linker, and
B is a targeting moiety capable of binding a first tumor-associated antigen expressed by at least some cells in the tumor;
with the proviso that if A-L- is a metal complex of Compound 1 as shown below, then B is not AVE1642 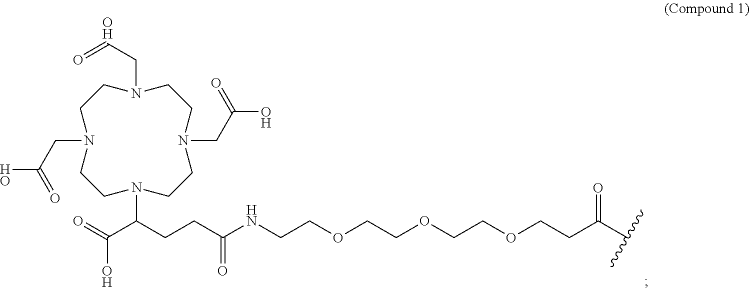
wherein said administering of said radioimmunoconjugate results in infiltration of a CD8⁺ T cell population into the core of the tumor;
wherein said CD8⁺ T cell population comprises CD8+ T cells expressing a T-cell receptor (TCR) specific for a second tumor-associated antigen expressed by at least some cells in the tumor; and
wherein the CD8+ T cell is capable of preferentially killing a cell expressing the second tumor-associated antigen.

2. The method of claim 1, wherein said CD8+ T cell population is detectable in the core of the tumor at a level greater than a reference level.

3. The method of claim 2, wherein said CD8+ T cell population is detectable at a level at least two-fold greater than the reference level.

4. The method of claim 3, wherein said CD8+ T cell population is detectable at a level at least three-fold greater than the reference level.

5. The method of claim 4, wherein said CD8+ T cell population is detectable at a level at least four-fold greater than the reference level.

6. The method of claim 5, wherein said CD8+ T cell population is detectable at a level at least five-fold greater than the reference level.

7. The method of claim 1, wherein said CD8+ T cell population represents at least 5% of cells in the core of the tumor.

8. The method of claim 7, wherein said CD8+ T cell population represents at least 7.5% of cells in the core of the tumor.

9. The method of claim 8, wherein said CD8+ T cell population represents at least 10% of cells in the core of the tumor.

10. The method of claim 9, wherein said CD8+ T cell population represents at least 12.5% of cells in the core of the tumor.

11. The method of claim 10, wherein said CD8+ T cell population represents at least 15% of cells in the core of the tumor.

12. The method of claim 1, wherein said CD8+ T cells represent at least 15% of said CD8+ T cell population.

13. The method of claim 12, wherein said CD8+ T cells represent at least 20% of said CD8+ T cell population.

14. The method of claim 13, wherein said CD8+ T cells represent at least 25% of said CD8+ T cell population.

15. The method of claim 14, wherein said CD8+ T cells represent at least 30% of said CD8+ T cell population.

16. The method of claim 15, wherein said CD8+ T cells represent at least 35% of said CD8+ T cell population.

17. The method of claim 16, wherein said CD8+ T cells represent at least 40% of said CD8+ T cell population.

18. The method of claim 17, wherein said CD8+ T cells represent at least 45% of said CD8+ T cell population.

19. The method of claim 18, wherein said CD8+ T cells represent at least 50% of said CD8+ T cell population.

20. The method of claim 19, wherein said CD8+ T cells represent at least 55% of said CD8+ T cell population.

21. The method of claim 20, wherein said CD8+ T cells represent at least 60% of said CD8+ T cell population.

22. The method of claim 21, wherein said CD8+ T cells represent at least 65% of said CD8+ T cell population.

23. The method of claim 22, wherein said CD8+ T cells represent at least 70% of said CD8+ T cell population.

24. The method of any one of claims 1-23, wherein said CD8+ T cells are detectable in the subject at least 10 days after the step of administering.

25. The method of claim 24, wherein said CD8+ T cells are detectable in the subject at least 15 days after the step of administering.

26. The method of claim 25, wherein said CD8+ T cells are detectable in the subject at least 20 days after the step of administering.

27. The method of claim 26, wherein said CD8+ T cells are detectable in the subject at least 25 days after the step of administering.

28. The method of claim 27, wherein said CD8+ T cells are detectable in the subject at least 30 days after the step of administering.

29. The method of claim 28, wherein said CD8+ T cells are detectable in the subject at least 40 days after the step of administering.

30. The method of any one of claims 1-29, wherein the first tumor-associated antigen is different than the second tumor-associated antigen.

31. The method of claim 30, wherein the second tumor-associated antigen is a neoantigen.

32. The method of any one of claims 1-31, wherein the tumor is a primary tumor.

33. The method of any one of claims 1-31, wherein the tumor is a secondary tumor.

34. The method of any one of claims 1-33, wherein the tumor is not highly immunogenic.

35. The method of claim 34, wherein the tumor is immunologically cold.

36. The method of any one of claims 1-35, wherein the tumor is at least 100 mm3 in volume at the time of administering.

37. The method of claim 36, wherein the tumor is at least 150 mm3 in volume at the time of administering.

38. The method of claim 37, wherein the tumor is at least or about 175 mm3 in volume at the time of administering.

39. The method of any one of claims 1-38, wherein the tumor is a solid tumor.

40. The method of claim 39, wherein the solid tumor is a sarcoma.

41. The method of claim 40, wherein the sarcoma is selected from the group consisting of angiosarcoma or hemangioendothelioma, astrocytoma, chondrosarcoma, Ewing's sarcoma, fibrosarcoma, glioma, leiomyosarcoma, liposarcoma, malignant fibrous histiocytoma (MFH), mesenchymous or mixed mesodermal tumor, mesothelial sarcoma or mesothelioma, myxosarcoma, osteosarcoma, rhabdomyosarcoma, and synovial sarcoma.

42. The method of claim 41, wherein the sarcoma is osteosarcoma.

43. The method of claim 39, wherein the solid tumor is a carcinoma.

44. The method of claim 43, wherein the carcinoma is selected from the group consisting of adenoid cystic carcinoma, adrenocortical carcinoma, bladder cancer, breast cancer, cervical cancer, colorectal cancer, endometrial cancer, gallbladder carcinoma, gastric cancer, head and neck cancer, lung cancer (e.g., small cell lung cancer or non-small cell lung cancer, or adenocarcinoma of the lung), neuroblastoma, neuroendocrine cancer, ovarian cancer, pancreatic cancer, prostate cancer, renal cancer, testicular cancer.

45. The method of claim 44, wherein the carcinoma is bladder cancer.

46. The method of claim 44, wherein the carcinoma is pancreatic cancer.

47. The method of claim 44, wherein the carcinoma is breast cancer.

48. The method of claim 44, wherein the carcinoma is head and neck cancer.

49. The method of claim 44, wherein the carcinoma is liver cancer.

50. The method of claim 44, wherein the carcinoma is lung cancer.

51. The method of claim 44, wherein the carcinoma is a brain cancer.

52. The method of claim 44, wherein the carcinoma is neuroblastoma.

53. The method of claim 44, wherein the carcinoma is melanoma.

54. The method of any one of claims 1-38, wherein the tumor is a liquid tumor.

55. The method of any one of claims 1-54, wherein said step of administering results in inhibition of cell proliferation in the core of the tumor.

56. The method of any one of claims 1-55, wherein said step of administering results in slowing or inhibiting progression of the tumor.

57. The method of claim 56, wherein said step of administering results in regression of the tumor.

58. The method of claim 57, wherein said step of administering results in complete regression of the tumor.

59. The method of any one of claims 1-58, wherein said step of administering prevents or inhibits metastasis of tumor cells.

60. The method of any one of claims 1-59, wherein A-L- is a metal complex of a compound selected from the group consisting of

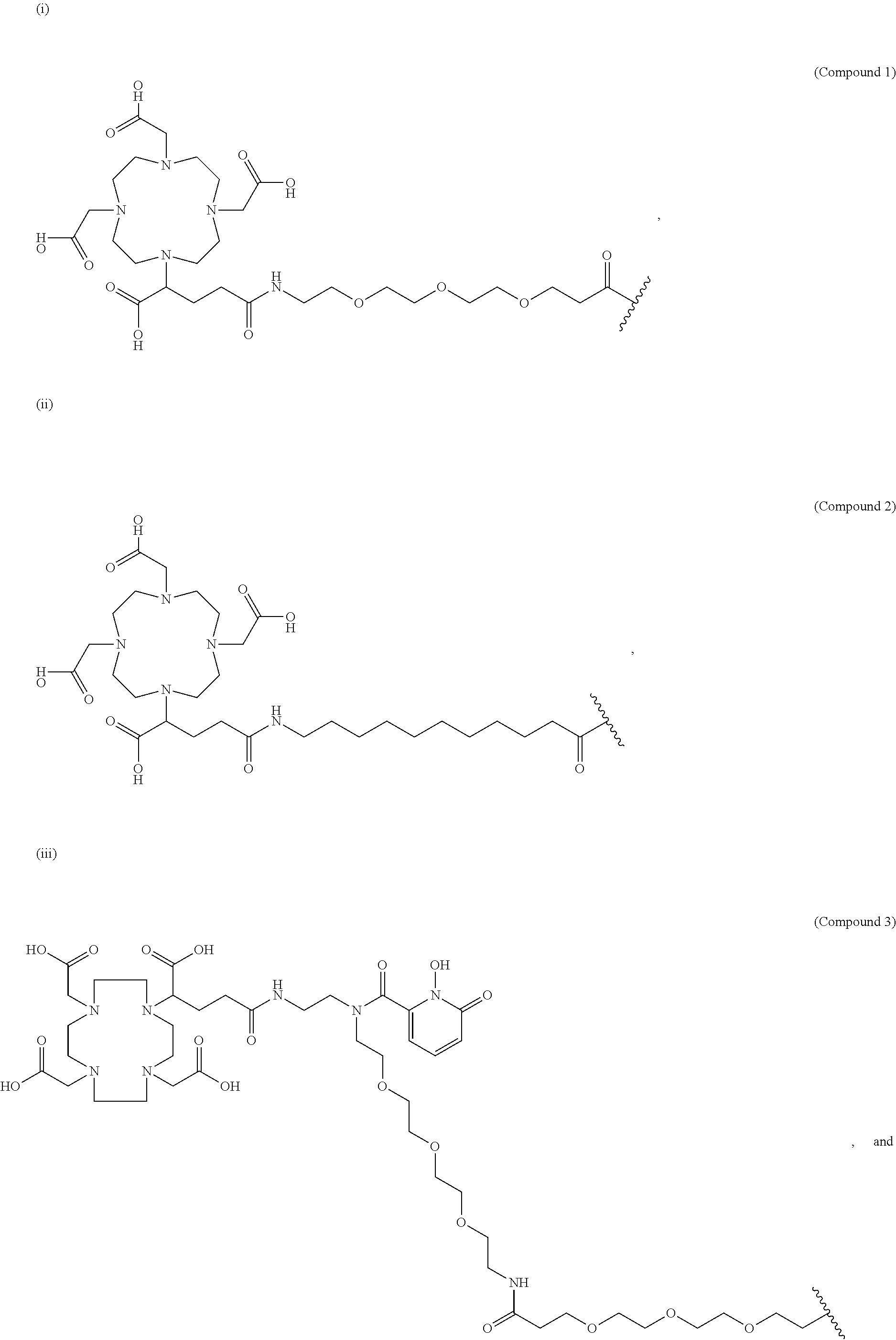 [SMILES: *C(=O)CCOCCOCCOCCNC(=O)CCC(C(=O)O)N1CCN(CC(=O)O)CCN(CC(=O)O)CCN(CC(=O)O)CC1 ; *C(=O)CCCCCCCCCCNC(=O)CCC(C(=O)O)N1CCN(CC(=O)O)CCN(CC(=O)O)CCN(CC(=O)O)CC1 ; *CCOCCOCCOCCC(=O)NCCOCCOCCOCCN(CCNC(=O)CCC(C(=O)O)N1CCN(CC(=O)O)CCN(CC(=O)O)CCN(CC(=O)O)CC1)C(=O)c1cccc(=O)n1O]

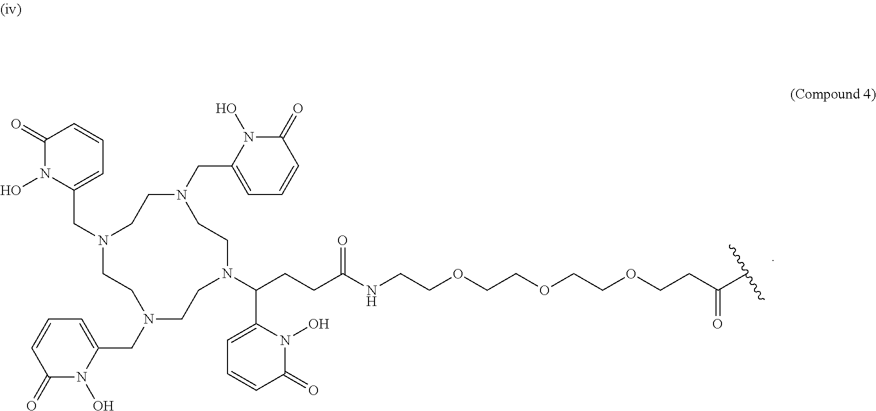 [SMILES: *C(=O)CCOCCOCCOCCNC(=O)CCC(c1cccc(=O)n1O)N1CCN(Cc2cccc(=O)n2O)CCN(Cc2cccc(=O)n2O)CCN(Cc2cccc(=O)n2O)CC1]

61. The method of any one of claims 1-60, wherein L has the structure -L¹-(L²)ₙ-, as shown within Formula I-b:
A-L¹-(L²)ₙ-B Formula I-b
wherein
A is a metal complex of chelating moiety, wherein the metal complex comprises a Actinium-225 (²²⁵Ac) or a progeny thereof,
B is a targeting moiety;
L¹ is optionally substituted C₁-C₆ alkyl, optionally substituted C₁-C₆ heteroalkyl, or optionally substituted aryl or heteroaryl;
n is 1-5; and
each L², independently, has the structure:
(—X¹-L³-Z¹—) Formula III
wherein
X is C═O(NR¹), C═S(NR¹), OC═O(NR¹), NR¹C═O(O), NR¹C═O(NR¹), —CH₂PhC═O(NR¹), —CH₂Ph(NH)C═S(NR¹), O, or NR¹; and each R¹ independently is H, optionally substituted C₁-C₆ alkyl, optionally substituted C₁-C₆ heteroalkyl, or optionally substituted aryl or heteroaryl, in which C₁-C₆ alkyl can be substituted by oxo (═O), heteroaryl, or a combination thereof;
L³ is optionally substituted C₁-C₅₀ alkyl or optionally substituted C₁-C₅₀ heteroalkyl; and
Z¹ is CH₂, C═O, C═S, OC═O, NR¹C═O, or NR¹, wherein R¹ is a hydrogen or optionally substituted C₁-C₆ alkyl or pyrrolidine-2,5-dione.

62. The method of claim 61, wherein the radioimmunoconjugate comprises the following structure: 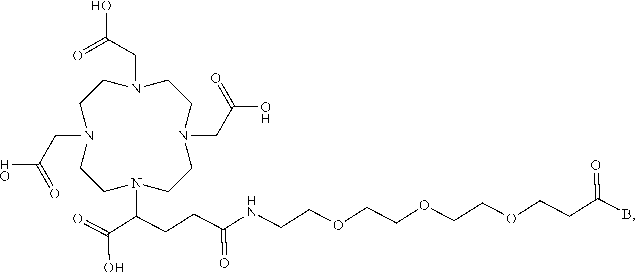
wherein B is the targeting moiety.

63. The method of any one of claims 1-62, wherein the targeting moiety comprises a polypeptide.

64. The method of any one of claims 1-63, wherein the targeting moiety comprises an antibody or an antigen-binding fragment thereof.

65. The method of any one of claims 1-64, wherein the targeting moiety has a molecular weight of at least 100 kDa.

66. The method of claim 65, wherein the targeting moiety has a molecular weight of at least 125 kDa.

67. The method of claim 66, wherein the targeting moiety has a molecular weight of at least 150 kDa.

68. The method of any one of claims 1-62, wherein the targeting moiety is a small molecule.

69. The method of any one of claims 1-68, wherein the first tumor-associated antigen is selected from the group consisting of Insulin-like Growth Factor 1 Receptor (IGF-1R), tumor epithelial marker-1 (TEM-1), and Fibroblast Growth Factor Receptor 3 (FGFR3).

70. The method of any one of claims 1-69, wherein the subject is a mammal.

71. The method of claim 70, wherein the subject is a human.

72. The method of any one of claims 1-71, wherein the subject is in need of treatment or prevention of cancer.

73. The method of claim 72, wherein the subject is diagnosed as having cancer.

74. The method of any one of claims 1-73, wherein the subject is in need of treatment of a refractory cancer.

75. The method of any one of claims 1-74, wherein the step of administering comprises systemic administration of the radioimmunoconjugate.

76. The method of claim 75, wherein systemic administration comprises parenteral administration.

77. The method of claim 76, wherein parenteral administration comprises intravenous administration.

78. The method of claim 76, wherein parenteral administration comprises intraarterial administration.

79. The method of claim 76, wherein parenteral administration comprises intraperitoneal administration.

80. The method of claim 76, wherein parenteral administration comprises subcutaneous administration.

81. The method of claim 76, wherein parenteral administration comprises intradermal administration.

82. The method of claim 75, wherein systemic administration comprises enteric administration.

83. The method of claim 82, wherein enteric administration comprises trans-gastroenteric administration.

84. The method of claim 82, wherein enteric administration comprises oral administration.

85. The method of any one of claims 1-84, wherein the step of administering comprises local administration of the radioimmunoconjugate.

86. The method of claim 85, wherein local administration comprises peritumoral injection.

87. The method of claim 85, wherein local administration comprises intratumoral injection.

88. The method of any one of claims 1-87, wherein the step of administering comprises contacting, ex vivo, the radioimmunoconjugate with a body fluid of said subject, wherein said body fluid contains at least one cancer cell.

89. The method of any one of claims 1-88, wherein the radioimmunoconjugate is not administered in combination with another cytotoxic agent.

90. The method of any one of claims 1-89, said method further comprising administering to the subject an additional therapeutic agent after the step of administering the radioimmunoconjugate.

91. The method of claim 90, wherein the additional therapeutic agent is a non-cytotoxic agent.

92. The method of claim 90 or 91, wherein the radioimmunoconjugate is administered in a lower effective dose.

93. The method of claim 90, 91, or 92 wherein the additional therapeutic agent is administered in a lower effective dose.
